(19) United States
(12) Patent Application Publication
(10) Pub. No.: US 2025/0250325 A1
(43) Pub. Date: Aug. 7, 2025

(54) Methods of Manufacturing Alpha-1 Antitrypsin Compositions For Treatment of Disease Disorders

(71) Applicants: Darren Lezdey, Daytona Beach, FL (US); Jarett Lezdey, Daytona Beach, FL (US); Walter Gahagan, Glen Cove, NY (US)

(72) Inventors: Darren Lezdey, Daytona Beach, FL (US); Jarett Lezdey, Daytona Beach, FL (US); Walter Gahagan, Glen Cove, NY (US)

(21) Appl. No.: 18/445,789

(22) Filed: Feb. 2, 2024

(51) Int. Cl. C07K 14/81; C12P 21/00

(52) U.S. Cl. CPC C07K 14/81; C12P 21/005

(57) ABSTRACT

A method for producing recombinant glycoproteins, focusing on human alpha-1 antitrypsin (AAT), using Pichia Pastoris yeast. Involves expressing the AAT polypeptide in an engineered yeast strain lacking the wild-type OCH1 gene and containing either a mutant OCH1 gene or a mannosidase gene. After expression, the AAT-containing supernatant is isolated, and the non-natively glycosylated AAT is purified to achieve a substantially homogeneous population of N-glycans. The purification process aims to limit the presence of specific glycan structures to less than 20%. Throughout the manufacturing process, including isolation and purification, the vitality of the Pichia Pastoris yeast strain is carefully maintained. This method offers precise control over glycosylation patterns, ensuring the production of recombinant glycoproteins with tailored characteristics for diverse biotechnological and therapeutic applications.

### CROSS-REFERENCE TO RELATED APPLICATIONS

This application is a continuation-in-part application of U.S. application Ser. No. 16/980,238, filed Sep. 11, 2020, now abandoned, which claims priority of International Patent Application No. PCT/US2019/021642, filed Mar. 11, 2019, which claims priority of U.S. Provisional Application No. 62/641,752, filed Mar. 12, 2018, all of which are incorporated by reference in their entities.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on May 16, 2024 is entitled “Sequence_Listing_3050_2_PCT_US_ST26.xml”, and is 48,253 bytes in size.

### BACKGROUND OF INVENTION

The present invention relates to methods and compositions directed at obtaining a nonnatively glycosylated recombinant human alpha-1 antitrypsin (A1AT) peptides that are glycosylated in a non-native configuration that confer enhanced biologic activities. Alpha-1 antitrypsin or α1-antitrypsin (A1AT or A1AT) is a protease inhibitor belonging to the serpin superfamily. It has also been referred to as serum trypsin inhibitor and alpha-1 proteinase inhibitor (A1PI), because it inhibits a wide variety of proteases. Inflammatory conditions often induce an acute-phase response and the concentration of alpha-1-antitripsin is substantially increased. Alpha-1 antitrypsin deficiency (α1-antitrypsin deficiency, A1AD) is a genetic disorder that causes defective production of alpha-1 antitrypsin (A1AT), leading to decreased A1AT activity in the blood and lungs, and deposition of excessive abnormal A1AT protein in liver cells resulting in respiratory complications such as emphysema, or COPD (chronic obstructive pulmonary disease) in adults and cirrhosis in adults or children. Current therapy for alpha-1 antitrypsin deficiency associated lung disease is augmentation or replacement therapy with alpha-1 antitrypsin protein (A1AT) from the blood plasma of healthy human donors to augment (increase) the alpha-1 levels circulating in the blood and lungs. Lung-affected A1AT patients can receive intravenous infusions of therapeutic concentrations of products derived from human plasma of blood donors.

Approved products include Prolastin-CR, Aralast NP™, Zemaira®, Glassia® or Trypsone®. Such augmentation infusions must typically be given by healthcare professionals in the home, at a physician's office, outpatient infusion center or other medical facility by weekly intravenous infusion and are ongoing and lifelong. Thus, current augmentation therapy is very expensive to obtain and administer. As human blood products, augmentation therapy also carries the risk of transmitting infectious agents (e.g., viruses, prion diseases (such as, variant Creutzfeldt-Jakob disease (vCJD)). There is, therefore, a longstanding and unmet need for a safe and affordable source of A1AT product that has the immunomodulatory activity of native A1AT. Described herein are just such compositions and methods of making them for subsequent use as effective therapeutics.

### SUMMARY OF THE INVENTION

The present invention provides a continuous method of making a recombinant alpha-1 antitrypsin glycoprotein having non-native glycosylation, utilizing spliced human DNA into a yeast cell to effectuate extracellular expression, which is inherent in the teachings of the parent applications of this application. The extracellular expression is a continuous process and allows for continuous production in the present invention process for two reasons: (1) unlike other processes, the recovery of the end product does not involve the destruction (killing) of the yeast, and (2) the yeast is self-generating, i.e., it continues its growth and its extracellular expression in its production environment.

In one embodiment of the present invention manufacturing process, the invention continuously provides a recombinant A1AT glycoprotein including human alpha-1 antitrypsin having non-native glycosylation. In one embodiment, the recombinant A1AT includes a population of substantially homogeneous N-glycans. In various of these embodiments, the population of N-glycans is more than about 40% homogenous, more than about 50% homogenous, more than about 60% homogenous, more than about 70% homogenous, more than about 80% homogenous, more than about 90% homogenous, or more than about 95% homogenous.

In some preferred embodiments of the present invention, the method of manufacturing recombinant glycoprotein comprising human alpha-1 antitrypsin having non-native glycosylation with a substantially homogeneous population of N-glycans, in an extracellular expression process includes: (a) expressing a polynucleotide sequence encoding human alpha-1 antitrypsin polypeptide in an engineered AAT expression strain comprising one of a mutant OCH1 gene and a mannosidase gene, wherein the engineered AAT expression strain does not contain a WT OCH1 gene wherein the AAT expression strain is an expression vector that is a Pichia Pastoris Yeast strain; (b) isolating supernatant; and (c) purifying the non-natively glycosylated AAT polypeptide from the supernatant to provide the recombinant glycoprotein and less than 20% Man7GlcNAc2, Man8GlcNAc2 and Man9GlcNAc2, wherein the method of manufacturing, including the isolating and the purifying, is performed while maintaining the vitality of the Pichia Pastoris Yeast. In some of these embodiments, the glycoprotein is SEQ ID NO: 18. In some of these embodiments, the non-native glycosylation comprises a population of at least 80% homogeneous N-glycans. In some of these embodiments, the Man7GlcNAc2, Man8GlcNAc2, and Man9GlcNAc2 N-glycans comprise less than about 10% of the total N-glycans; more preferably, the Man7GlcNAc2. Man8GlcNAc2, and Man9GlcNAc2 N-glycans comprise less than about 5% of the total N-glycans and most preferably, the Man7GlcNAc2, Man8GlcNAc2, and Man9GlcNAc2 N-glycans comprise less than about 1% of the total N-glycans.

In another preferred embodiment of the present invention, the method of manufacturing recombinant glycoprotein comprising human alpha-1 antitrypsin having non-native glycosylation with a substantially homogeneous population of N-glycans, in an extracellular expression process includes: (a) in a continuous flow process, expressing a polynucleotide sequence encoding human alpha-1 antitrypsin polypeptide in an engineered AAT expression strain comprising one of a mutant OCH1 gene and a mannosidase gene, wherein the engineered AAT expression strain does not contain a WT OCH1 gene wherein the AAT expression strain is an expression vector that is a Pichia Pastoris Yeast strain; (b) isolating supernatant; and (c) purifying the non-natively glycosylated AAT polypeptide from the supernatant to provide the recombinant glycoprotein product with less than 20% Man7GlcNAc2, Man8GlcNAc2 and Man9GlcNAc2, wherein the method of manufacturing, including the isolating and the purifying, is performed while maintaining the vitality of the Pichia Pastoris Yeast. As well as stated above, the preferred embodiments here include wherein the glycoprotein is SEQ ID NO: 18; include wherein the non-native glycosylation comprises a population of at least 80% homogeneous N-glycans; include wherein Man7GlcNAc2, Man8GlcNAc2, and Man9GlcNAc2 N-glycans comprise less than about 10% of the total N-glycans; include wherein Man7GlcNAc2, Man8GlcNAc2, and Man9GlcNAc2 N-glycans comprise less than about 5% of the total N-glycans; and include wherein Man7GlcNAc2, Man8GlcNAc2, and Man9GlcNAc2 N-glycans comprise less than about 1% of the total N-glycans.

In some of these continuous flow present invention methods, the manufacturing is performed in at least one reactor wherein the reactor includes feed lines and output lines, and output is released in a steady flow, for subsequent product purification and isolation. In some of these continuous flow present invention methods, the manufacturing is performed in at least one reactor wherein the reactor includes feed lines and output lines, and output is released in a steady flow, for subsequent product purification and isolation and feed lines include continuous feed of at least the engineered AAT expression strain.

In some of these continuous flow present invention methods, the method of manufacturing is performed in at least one reactor wherein the reactor includes feed lines and output lines, and output is released in a steady flow, for subsequent product purification and isolation and feed lines include continuous feed of at least the engineered AAT expression strain and a separate continuous feed of the Pichia Pastoris Yeast.

In some of these continuous flow present invention methods, the manufacturing is performed in at least one reactor wherein the reactor includes feed lines and output lines, and output is released in a steady flow, for subsequent product purification and isolation and feed lines include continuous feed of at least the engineered AAT expression strain and output lines include purification and isolation of the recombinant glycoprotein product, wherein output Pichia Pastoris Yeast is at least in part recycled back to the reactor.

In some of these continuous flow present invention methods, the manufacturing is performed in at least one reactor wherein the reactor includes feed lines and output lines, and output is released in a steady flow, for subsequent product purification and isolation and feed lines include continuous feed of at least the engineered AAT expression strain and a separate continuous feed of the Pichia Pastoris Yeast, and output lines include purification and isolation of the recombinant glycoprotein product, wherein output Pichia Pastoris Yeast is at least in part recycled back to the reactor.

In one embodiment, the population of N-glycans comprises, consists essentially of, or consists of one or more of Man5GlcNAc2, Man6GlcNAc2, Man7GlcNAc2, Man8GlcNAc2, Man9GlcNAc2, GlcNAcMan5GlcNAc2, GalGlcNAcMan5GlcNAc2, GalGlcNAcMan3GlcNAc2, GlcNAcMan3GlcNAc2, GlcNAc2Man3GlcNAc2, Gal2GlcNAc2Man3GlcNAc2, Hex 10GlcNac2, Hex11GlcNac2, Hex 12GlcNac2, Hex 13GlcNac2, Hex 14GlcNac2, Hex 15GlcNac2, and Hex 16GlcNac2.

In one embodiment, the population of N-glycans comprises, consists essentially of, or consists of one or more of Man5GlcNAc2, Man8GlcNAc2, GlcNAcMan5GlcNAc2, GalGlcNAcMan5GlcNAc2, GalGlcNAcMan3Glc.NAc2, GlcNAcMan3GlcNAc2, GlcNAc2Man3GlcNAc2, and Gal2GlcNAc2Man3GlcNAc2.

In one embodiment, the population of N-glycans comprises, consists essentially of, or consists of one or more of ManSGlcNAc2, Man7GlcNAc2, Man8GlcNAc2, Man9GlcNAc2, Hex 10GlcNac2, Hex11GlcNac2, Hex 12GlcNac2, Hex 13GlcNac2, Hex 14GlcNac2-Hex 15GlcNac2, and Hex 16GlcNac2.

In one embodiment, the population of N-glycans comprises, consists essentially of, or consists of one or more of Man5GlcNAc2, Man8GlcNAc2, Man9GlcNAc2, Hex 10GlcNac2, Hex 11GlcNac2, Hex 12GlcNac2, Hex 13GlcNac2, Hex 15GlcNac2, and Hex 16GlcNac2. In one embodiment, the population of Man7GlcNAc2, Man8GlcNAc2, and Man9GlcNAc2 N-glycans includes less than 20%, less than about 10%, less than about 5%, or less than about 1% of the total N-glycans. In one embodiment, the population of Hex 10GlcNac2, Hex11GlcNac2, Hex 12GlcNac2, Hex 13GlcNac2, Hex 15GlcNac2, and Hex 16GlcNac2 N-glycans comprise less than about 20%, less than about 10%, less than about 5%, or less than about 1% of the total N-glycans. In one embodiment, the population of HexnGlcNac2 N-glycans comprise less than about 20%, less than about 10%, less than about 5%, or less than about 1% of the total N-glycans, where n is 1-25.

In one embodiment, the recombinant glycoprotein has increased alpha-1 antitrypsin biological activity as compared to the activity of purified natively glycosylated alpha-1 antitrypsin protein. The biological activity has at least about 10% more, at least about 20%, at least about 50% more, at least about 100% more, or at about least 200% more alpha-1 antitrypsin biological activity as compared to the activity of purified natively glycosylated alpha-1 antitrypsin protein. In one embodiment, the purified natively glycosylated alpha-1 antitrypsin protein is derived from human blood. In one embodiment, the biological activity is characterized as a reduction in the activity level of one or more of MCP-1, IL-1, IL-6, or MMP9.

In one embodiment, the present invention provides a method of treating or preventing an alpha-1 antitrypsin mediated lung or topical disease or disorder in a subject, including administering an effective amount of the recombinant glycoprotein disclosed herein. In one embodiment, the alpha-1 antitrypsin mediated lung or topical disease or disorder includes, but is not limited to, chronic obstructive pulmonary disease (COPD), asthma, bronchiectasis, and emphysema. This would include Sars COV-2 the infectious disease that causes Covid 19 and any future variants including a mixture of Covid 19 and a vast reservoir of viruses circulating among wildlife that can become a new source of infectious disease to which humans do not have immunity. Such as Zika, Ebola and Sars virus among others. These all share a commonality of causing inflammation and or attacking human respiratory system and lymph nodes resulting in death or severe health issues.

In one embodiment, the alpha-1 antitrypsin mediated lung or topical disease or disorder includes, but is not limited to, eye and ear otitis media otitis externa, arthritis, conjunctivitis, hot spots, atopic dermatitis, skin wound, pruritus, skin inflammation, psoriasis, and mast cell tumors.

In one embodiment, the present invention provides a recombinant A1AT glycoprotein prepared by a process including the steps of: (a) expressing a polynucleotide sequence encoding human alpha-1 antitrypsin polypeptide in an engineered A1AT expression strain includes at least one of a mutant OCH1 gene and a mannosidase gene, wherein the engineered A1AT expression strain does not contain a WT OCH1 gene; (b) isolating supernatant; and (c) purifying the non-natively glycosylated A1AT polypeptide from the supernatant to provide the recombinant glycoprotein disclosed herein. Any embodiment discussed with respect to one aspect of the invention applies to other aspects of the invention as well.

The embodiments in the Example section are understood to be embodiments of the invention that are applicable to all aspects of the invention. Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention can be better understood by reference to one or more of these drawings in combination with the detailed description of specific embodiments presented herein.

FIG. 1: Depicts a PAGE A1AT expression in SuperMan5 cells: initial testing of A1AT clones. Illustrates 8% NuPAGE/MES separation and SYPRO Ruby protein staining of 10 μl of supernatant from different A1AT SuperMan5 expression clones as compared at 36 hr post Methanol induction (pMi). Groupings represent proteins expressed in: (2) bG Yeast1000144 strain which contains 2 copies (D); (1) bG Yeast-100127 strain which contains a single copy(S); or (3) parental strain, SuperMan5 (+) without expression cassette (negative control). Lanes marked (M) contain 2 μl BioRad Precision Plus unstained protein standards, kDA. The expression cassettes were: one containing the ORF sequence (SEQ ID NO: 6) designated as (hA1AT) and was sub cloned from a pPICZalphaA-A1AT expression vector created at Invitrogen; a second containing the ORF sequence (SEQ ID NO: 10) that contained an A1AT ORF codon that had been optimized for expression in Pichia designated as DNA2-ORF (aKR) (A) (2A1AT); and a third ORF sequence (SEQ ID NO: 11) designated as DNA2-ORF+HisTag, (aKR) (H) (2tA1AT), which contains an added N-term HisTag.

FIG. 2: Depicts a PAGE of A1AT expression under different expression conditions. Shake Flask Optimization: A1AT expression at later time-points with select clones shows 8% NuPAGE/MES separation and SYPRO Ruby protein stain of shake flask cultures of select different SuperMan5-A1AT expression clones at 60 hr post Methanol induction (pMi) and 36 hr pMi. Samples from two copy clones are shown on the gel on the left and in lane 1: ID2-2 at 60 hr pMi; lane 2: ID2-1 at 60 hr pMi; lane 3: ID2-1 at 36 hr pMi; lane 4:2 μl of marker BioRad Precision Plus unstained protein standards. Samples from single copy clones are shown on the gel on the right and in lane 1:2 μl of marker BioRad Precision Plus unstained protein standards; lane 2: IS1-1 at 36 hr pMi; lane 3: IS1-1 at 60 hr pMi; lane 4: IS1-2 at 60 hr pMi.

FIG. 3: Depicts AAT-Elastase binding assay. Purified A1AT with and without Elastase: shows a 8% NuPAGE/MES separation and SYPRO Ruby protein stain of lanes containing 10 μl of induced rALAT supernatant with or without elastase (lane 1); with 63 ng of elastase (lane 2); with 125 ng of elastase (lane 3); with 250 ng of elastase (lane 4); with 500 ng of elastase (lane 5). SM5 parent strain supernatant with 500 ng of elastase (lane 6); hT2 (an unrelated control protein) with 500 ng of elastase (lane 7).

FIG. 4: Depicts A1AT expression under different methanol conditions. Optimization of methanol induction conditions shows an 8% NuPAGE/MES separation and SYPRO Ruby protein stain of lanes containing 10 μl of induced ID2-1 nAAT SuperMan5 strain at different time points following induction with methanol and under different conditions. Lane 1 contains only 5 μl of the sample shown in lane 5; lane 2 contains 2 μl of marker BioRad Precision Plus unstained markers; lane 3 contains dilute culture 24 hr pMi; lane 4 contains dilute culture 36 hr pMi; lane 5 contains undiluted culture supernatant 24 h pMi; lane 6 contains undiluted culture supernatant 36 hr pMi; lane 7 contains dilute culture 48 hr pMi; lane 8 contains undiluted culture supernatant from 2nd 24 hr pMi sample; lane 9 contains 6 samples from BMY culture 12 hr pMi; lane 10 contains sample from RSM culture 12 hr pMi; lane 11 contains sample from extra A1AT culture 12 hr pMi; lane 12 contains sample from the first 2 ml ID2-1G induction; lane 13 contains RT gly gD (+control, hT2); lane 14 contains 3/16 gD (+control, hT2); and lane 15 contains the same 2 μl of BioRad Precision Plus markers as are in lane 2.

FIG. 5: Depicts a comparison of Methanol induced supernatant samples by PAGE.

FIG. 6: Depicts a PAGE analyses after Tangential Flow Filtration (TFF): shows an 8% polyacrylamide gel electrophoresis (PAGE)/MES (NuPAGE® SDS-PAGE: Thermo Fisher Scientific) that has been stained with SYPRO Ruby (Molecular Probes). Lane M contains 2 μl of BioRad Precision Plus unstained standard protein markers (kDA: the 50 kDa band contains approximately 90 ng of protein); Lane 1 contains a sample obtained from bottle 1 (approximately 1.2 liters) of supernatant prior to TFF (LF-B); Lane 2 contains a sample obtained from bottle 2 (approximately 1.2 liters) of supernatant prior to TFF (MD-B); Lane 3 contains a sample obtained from final A-1 induction (2nd, approx. 36 hrs pMi); Lane 4 contains a sample obtained from final A-mix induction (2nd, approx. 36 hrs p.Mi); Lane 5 contains a sample obtained from final, dilute induction OD 1.5 to 13 on methanol; Lane 6 contains a sample obtained from stratum permeate #1 (from concentration); Lane 7 contains a sample obtained from stratum permeate #2 (from concentration); Lane 8 contains a sample obtained from stratum permeate #3 (from concentration); Lane 9 contains a sample obtained from stratum permeate #4, gradient from buffer exchange, gradient in bottle taken from top without mixing; Lane 10 contains a sample obtained from the 2M NaCl eluate from the Q column; Lane 11 contains a 10 μl Diafiltered sample (BCA determination was 4.0 mg/ml) (approximately 200 ml of this concentrated/TFF); Lane 12 contains a 2 μl Diafiltered sample; Lane 13 contains a 0.4 μl Diafiltered sample; Lane 14 contains a 0.08 μl Diafiltered sample; Lane 15 contains a 1.25 μl Diafiltered sample incubated with 2 μg of Elastase (2 μl of 1 mg/ml stock mixed with rA1AT in 10 μl of PBS for 15 min at room temperature; Lane 16 contains a 2.5 μl Diafiltered sample incubated with 2 μg of Elastase as in lane 15; Lane 17 contains a 5 μl Diafiltered sample incubated with 2 μg of Elastase as in lane 15; Lane 18 contains a 10 μl Diafiltered sample incubated with 2 μg of Elastase as in 30 lane 15; and Lane 19 contains a 10 μl of the sample from lane 2 (MD-B) from the load incubated with 2 μg of Elastase as in lane 15.

FIG. 7: Depicts PAGE of A1AT protein in the flow through and eluted fraction of anion exchange columns. HiTrap Q initial fraction of 100 ml supernatant: shows an 8% polyacrylamide gel electrophoresis (PAGE)/MES (NuPAGE® SDS-PAGE: Thermo Fisher Scientific) that has been stained with SYPRO Ruby (Molecular Probes) of the flow through and HiTrap Q fractions from the initial application. Lane M contains 2.2 μl of BioRad Precision Plus unstained standard protein markers (kDA: the 50 kDa band contains approximately 100 ng of protein); Lane FT contains 2 μl of flow through from fractions A, B, C, and D the hydroxyapatite column; Lane WI contains 2 μl of buffer exchange permeate; the other lanes contain 2 μl of fractions 5-25 from the 2×5 ml hydroxyapatite anion exchange column.

FIG. 8: Depicts PAGE of A1AT protein in the flow through and eluted fraction of anion exchange columns. HiTrap Q second 100 ml fraction: shows an 8% polyacrylamide gel electrophoresis (PAGE)/MES (NuPAGE® SDS-PAGE: Thermo Fisher Scientific) that has been stained with SYPRO Ruby (Molecular Probes) of the flow through and HiTrap Q fractions from the original fractions from a 2nd HiTrap Q chromatography. Lane M contains 2.2 μl of BioRad Precision Plus unstained standard protein markers (kDA: the 50 kDa band contains approximately 100 ng of protein); Lane FT contains 2 μl of flow through from fractions A, B, C, and D the hydroxyapatite column; Lane WI contains 2 μl of buffer exchange permeate; the other lanes contain 2 μl of fractions 5-25 from the 2×5 ml hydroxyapatite anion exchange column.

FIG. 9: Depicts PAGE analysis of HiTrap Q fractions. Dilutions of the initial fractions (left), and the original fractions from a 2nd HiTrap Q chromatography: shows an 8% polyacrylamide gel electrophoresis (PAGE)/MES (NuPAGE® SDS-PAGE: Thermo Fisher Scientific) that has been stained with SYPRO Ruby (Molecular Probes). Lane M contains 2.2 μl of Bio-Rad Precision Plus unstained standard protein markers (kDA: the 50 kDa band contains approximately 100 ng of protein); Lane FT contains 2 μl of flow through from the hydroxyapatite column; Lane WI contains 2 μl of buffer exchange permeate; the other lanes contain 2 μl of fractions 5-27 from the hydroxyapatite column.

FIG. 10: Depicts PAGE analysis of Phenyl Sepharose fractions.

FIG. 11: Depicts a PAGE analysis of Hydroxyapatite fractions.

FIG. 12: Depicts a PAGE analysis of final purification fractions.

FIG. 13: Depicts a PAGE of purified A1AT, +/−Elastase.

FIG. 14: Depicts a Western analysis of purified A1AT, +/−Elastase (same sample as in FIG. 11).

FIG. 15: Depicts a Coomassie blue stained A1AT on PVDF membrane, just prior to excision for N-terminal sequencing.

FIG. 16: Depicts an analysis of purified A1AT after storage for 2 months at −80 and 4° C.

FIG. 17: Depicts stability testing of A1AT at 37° C., 30 hours.

FIG. 18: Stability testing of A1AT at 37° C., after 7 days.

FIG. 19: Depicts a PAGE of initial fermentation supernatants for A1AT production.

FIG. 20: Depicts a PAGE and Western analysis of Fermentation supernatants.

FIG. 21: Depicts a PAGE and Ruby staining of Fermentation supernatants.

FIG. 22: Illustrates Elastase activity studies: shown is the effect of native A1AT (Zemaira®) and recombinant rA1AT glycoprotein (at a final concentration of 0.08 mg/ml) on Elastase (at a concentration of 0.26 μM) activity.

FIG. 23: Illustrates Elastase complex formation: A1AT and rA1AT were incubated at a molar ratio of 1.2:1 in PBS with and without elastase. Incubation was performed at RT for 30 min and the sample was run in 7.5% SDS-PAGE gel. The gel was then stained with Coomassie blue R250 stain, distained with detaining solution (water: methanol: acetic acid ratio was 5:4:1). Lane M contains marker proteins; Lane 1 contains Elastase (mw=25 kDa) alone; Lane 2 contains Zemaira®, a native A1AT preparation; Lane 3 contains Zemaira® and Elastase, Lane 4 contains rA1AT recombinant A1AT glycoprotein with non-native glycosylation; Lane 5 contains rA1AT recombinant glycoprotein and elastase.

FIG. 24: Illustrates release of TNFα by human PBMC (peripheral blood mononuclear cells) cultures incubated for 24 hours following the addition of LPS, native A1AT (Zemaira®), recombinant rA1AT glycoprotein alone and in combination with LPS.

FIG. 25: Illustrates the release of lactate dehydrogenase (LDH) released into the media from damaged cells as a biomarker for cellular cytotoxicity and cytolysis resulting from exposure to recombinant rA1AT glycoprotein or native A1AT (Zemaira®).

FIG. 26: Illustrates the relative expression of IL-1B, TNFα mRNA levels in human PBMCs incubated overnight with recombinant rA1AT glycoprotein (0.05 mg/ml); LPS; the Docket No.: 2561-21P 9 combination of recombinant rA1AT glycoprotein (0.05 mg/ml) and LPS; and the combination of native A1AT (Zemaira®: 1 mg/ml) and LPS.

FIG. 27: Illustrates the results from ex vivo, mouse lung tissue treated for 24 hours with LPS (1000 ng/ml) alone or with addition of recombinant rA1AT glycoprotein (0.1 5 mg/ml) on the expression of IL-6, MMP9, MCP-1, and IL-1.

FIG. 28: Full MALDI TOF/TOF mass spectrum of N-linked glycans from 2016-01 ATT purified.

FIG. 29: Table of N-linked glycans from the glycoprotein detected by MALDI TOP/TOF MS.

### DETAILED DESCRIPTION OF THE INVENTION

The present invention provides, among other things, continuous process methods or making and for commercial manufacturing of recombinant alpha-1 antitrypsin glycoprotein having non-native glycosylation, via extracellular expression utilizing spliced human DNA into a controlled environment of yeast cells. A continuous process, as used herein, is one in which there is the flow of product on a continuous basis. In the context of the present invention methods of making, one embodiment involves utilizing yeast as a constituent wherein the yeast in not killed, to produce and procure the A1AT and/or rA1AT end product, and the yeast is thus permitted to live and to self-perpetuate. In some preferred embodiments, the yeast and the other constituents are fed at a controlled rate, into a reactor, wherein the product is recovered from outflow outside of the reactor. In other embodiments, the yeast and the other constituents are fed at a controlled rate, into a reactor, wherein the product is recovered from outflow outside of the reactor and the yeast product is isolated and is recycled back into the reactor. In yet other embodiments both the yeast and the other constituents are continuously fend into the reactor. In these present invention processes, the extracellular expression of the yeast enables the addition of other constituents in a timed flow process, such as for a continuous run, which may be for weeks or longer. Some manufacturing is contemplated within the scope of the present invention wherein continuous flow operation occurs for as long as the equipment and system hold up. Clearly, shutdowns are needed for maintenance, repairs, clean room updates and equipment replacement, but otherwise, the method is a continuous one wherein the reacted yeast maintains its vitality (is not killed) in the isolation process to procure product.

As used herein, Alpha-1 antitrypsin and α1-antitrypsin (A1AT or AAT) are used interchangeably. COMPOSITION Alpha-1 antitrypsin (A1AT) is a ˜52 kDa glycoprotein belonging to the serine protease inhibitor (serpin) superfamily. Examples of proteases inhibited by A1AT include neutrophil elastase (NE) in the lungs. In one embodiment, A1AT includes SEQ ID NO: 18, 20 SEQ ID NO: 19, or SEQ ID NO: 20. Human A1AT includes 3 N-linked glycans attached to asparagine residues 46, 83, and 247. Naturally occurring A1AT includes significant heterogeneity in glycosylation pattern and N-glycan structure. As such, several isoforms of A1AT exist, also known as glycoforms. A glycoform is an isoform of a protein that differs only with respect to the number or type of attached glycan. Glycoproteins often consist of a number of different glycoforms, with alterations in the attached saccharide or oligosaccharide. In one embodiment, the present invention provides a recombinant A1AT protein (rA1AT) having substantially homogeneous N-glycan structures.

By “substantially homogeneous” N-glycans it is meant that given a preparation containing a population of a particular glycoprotein of interest, at least about 40%, at least about 50%, at least about 60%, at least about 75%, at least about 80%, at least about 85%, at least about 90%, or at least about 95% of the N-glycans on the protein molecules within the population are the same. In one embodiment, the A1AT protein includes 1, 2, 3, 4, 5, 6, 7, 8, 9, 10, or 15 predominant N-glycan structures.

By “predominant N-glycan structure” or “predominant glycoform” it is meant a specific N-glycan structure or glycoform of (i.e., attached to) a protein constitutes the greatest percentage of all N-glycan structures or glycoforms of the protein. In certain specific embodiments, a predominant glycoform(s) accounts for at least about 40%, at least about 50%, at least about 60%, at least about 70%, at least about 80%, at least about 90%, or at least about 95% or greater of the population of all glycoforms on the protein. Examples of desirable N-glycan structures include, e.g., Man8GlcNAc2 (or “M8”) or Man5GlcNAc2 (“M5”). Additional desirable N-glycan structures include, GnM5 (GlcNAcMan5GlcNAc2), GalGnM5 (GalGlcNAcManSGlcNAc2), GalGnM3 (GalGlcNAcMan3GlcNAc2), GnM3 (GlcNAcMan3GlcNAc2), Gn2M3 (GlcNAc2Man3GlcNAc2), and Gal2Gn2M3 (Gal2GlcNAc2Man3GlcNAc2). The structures of these N-glycans have been described, e.g., in Jacobs et al., 2009, Nature Protocols 4:58-70, and are incorporated herein by reference. In one embodiment, the recombinant A1AT comprises, consists essentially of, or consists of one or more of the following glycans, Man5GlcNAc2, Man6GlcNAc2, Man7GlcNAc2, Man8GlcNAc2, Man9GlcNAc2, GlcNAcMan5GlcNAc2, GalGlcNAcMan5GlcNAc2, GalGlcNAcMan3Glc.NAc2, GlcNAcMan3GlcNAc2, GlcNAc2Man3GlcNAc2, Gal2GlcNAc2Man3GlcNAc2, Hex 10GlcNac2, Hex11GlcNac2, Hex 12GlcNac2, Hex 13GlcNac2, Hex 14GlcNac2, Hex 15GlcNac2, and Hex 16GlcNac2.

In another embodiment, the recombinant A1AT includes one or more of the following glycans, Man5GlcNAc2, Man8GlcNAc2, GlcNAcMan5GlcNAc2, GalGlcNAcMan5GlcNAc2, GalGlcNAcMan3GlcNAc2, GlcNAcMan3GlcNAc2, GlcNAc2Man3GlcNAc2, and Gal2GlcNAc2Man3GlcNAc2. In one embodiment, the A1AT comprises, consists essentially of, or consists of one or more of the following glycans, Man5GlcNAc2, Man7GlcNAc2, Man8GlcNAc2, Man9GlcNAc2, Hex 10GlcNac2, Hex11GlcNac2, Hex 12GlcNac2, Hex 13GlcNac2, Hex 14GlcNac2, Hex 15GlcNac2, and Hex 16GlcNac2. In one embodiment, the A1AT includes one or more of the following glycans, Man5GlcNAc2, Man8GlcNAc2, Man9GlcNAc2, Hex 10GlcNac2, Hex 11GlcNac2, Hex 12GlcNac2, Hex 13GlcNac2, Hex 15GlcNac2, and Hex 16GlcNac2. In another embodiment, Man7GlcNAc2, Man8GlcNAc2, and Man9GlcNAc2 N-glycans includes less than 20%, less than about 10%, less than about 5%, or less than about 1% of the total N-glycans of the recombinant A1AT disclosed herein. In another embodiment, Hex 10GlcNac2, Hex 11GlcNac2, Hex 12GlcNac2, Hex 13GlcNac2, Hex 15GlcNac2, and Hex 16GlcNac2 N-glycans include less than about 20%, less than about 10%, less than about 5%, or less than about 1% of the total N-glycans of the recombinant A1AT disclosed herein. In another embodiment, HexnGlcNac2 N-glycans include, individually or in combination, less than about 20%, less than about 10%, less than about 5%, or less than about 1% of the total N-glycans, where n is 1-25. In one embodiment, the recombinant A1AT glycoprotein disclosed herein includes substantially homogeneous N-glycan with Man5GlcNAc2 and Hex 10GlcNac2 in combination being the predominant N-glycan forms. In one embodiment, the recombinant A1AT glycoprotein includes substantially homogeneous N-glycan with Man5GlcNAc2 being the predominant N-glycan form.

In one embodiment, the predominant N-glycan form includes ManSGlcNAc2. To exemplify, Man5GlcNAc2 includes at least about 40% of the population of all glycoforms on the protein; Man5GlcNAc2 includes at least about 50% of the population of all glycoforms on the protein; Man5GlcNAc2 includes at least about 75% of the population of all glycoforms on the protein. In one embodiment, the population of N-glycans of recombinant rA1AT includes about 40-50%, about 40-60%, about 40-70%, about 40-80%, about 40-90%, about 50-70%, about 50-80%, about 60-90%, or about 70-90% of the Man5GlcNAc2. In one embodiment, the recombinant A1AT glycoprotein can be truncated at the Nterminus, C-terminus, or both. In one embodiment, the recombinant A1AT protein can be Cterminally truncated. In one embodiment, the C-terminal truncation includes truncation of 1, 2, 3, 4, 5, 6, or 7 C-terminal amino acids. In one embodiment, the α-terminal truncation includes loss of charged amino acid residues from the C-terminal 25 amino acid residues. In one embodiment, the C-terminal truncation includes loss of lysine from the c-terminal. In one embodiment, the recombinant A1AT protein can be n-terminally truncated. In one embodiment, the N-terminal truncation includes truncation of 1, 2, 3, 4, 5, 6, or 7 n terminal amino acids. In one embodiment, the n-terminal truncation includes loss of charged amino acid residues from the N-terminal amino acid residues. In one embodiment, deletion of glutamic and aspartic acid residues from the 1-50 N-terminal amino acids is contemplated. In one embodiment, the recombinant A1AT protein can contain both N-terminal and C-terminal truncations as described above. Functional equivalents of the rALAT protein have also been contemplated. As used herein, “functional equivalents” are to be understood as mutants that exhibit, in at least one of the sequence positions of human rA1AT protein, an amino acid substitution other than the one mentioned specifically, but still lead to a mutant which show the same or similar properties with respect to the wild type A1AT protein.

Functional equivalents include polypeptides having at least about 80%, at least about 85%, at least about 90%, or at least about 95% sequence identity to the human A1AT. It was unexpectedly discovered that the rA1AT protein described herein has increased biological activity as compared to the activity of purified natively glycosylated alpha-1 antitrypsin polypeptide. In one embodiment, the natively glycosylated alpha-1 antitrypsin polypeptide is purified from human blood. The biological activity of A1AT can be assessed by any method known in art. As used herein, biological activity of A1AT can be assessed in vitro or in vivo. In vitro bioactivity can be assessed by an elastase complex formation assay and cytotoxicity assay. These assays are commonly known in the art, and some of which are described herein. Those assays not described are incorporated herein by reference. In vitro methods included testing of supernatants of cell cultures from monocytes and neutrophils treated with LPS and/or A1AT and analyzed to determine levels of TNFα, IL-8, and IL-6.

In vivo methods included a mouse model for acute lung infection. In vivo, A1AT was shown to protect against TNFα or endotoxin-induced lethality, and in a mouse model of lung inflammation, A1AT was highly effective in suppressing Docket No.: 2561-21P 13 inflammation and connective tissue breakdown. Such in vivo biological activity testing is known in the art. In one embodiment, biological activity is characterized as a reduction in the activity level or serum level of one or more of MCP-1, IL-1, IL-6, MPO, and MMP9. In one embodiment, biological activity is characterized as a reduction in the activity level or serum level of one or more of IL-8 and TNFalpha.

In one embodiment, A1AT polypeptide having non-native glycosylation, as described herein, has at least about 10%, at least about 25%, at least about 50%, at least about 100%, or at least about 200%, more biological activity as compared to the activity of purified natively glycosylated alpha-1 antitrypsin. In one embodiment, A1AT polypeptide having non-native glycosylation, as described herein, has at least about 2-fold, at least about 5 fold, at least about 10 fold, at least about 20 fold, at least about 50 fold, or at least about 100 fold more biological activity as compared to the activity of purified natively glycosylated alpha-1 antitrypsin.

The suitability of rALAT glycoprotein compositions is readily determined by evaluation of its potency and selectivity using the methods described in this application and in the literature followed by evaluation of its toxicity, absorption, distribution, metabolism, excretion, or pharmacokinetics in accordance with standard pharmaceutical practice. In one embodiment, the present invention provides a method of producing the recombinant alpha-1 antitrypsin having non-native glycosylation disclosed herein.

The rA1AT can be produced by expression in a cell-based system and isolated there from suitable cell-based systems including bacterial systems. Examples of suitable bacterial systems include Escherichia coli, Corynebacterium, and Pseudomonas fluorescens. Further suitable cell-based systems include yeast systems such as Saccharomyces cerevisiae and Pichia Pastoris; Filamentous fungi; Baculovirus-infected cells; non-lytic insect cell expression; Leishmania; and Mammalian systems. Such systems are further described in Current Protocols in Molecular Biology (ISBN: 9780471142720). Modifications of the cells to affect the expressed rA1AT have been contemplated. Such modifications include expression of a mutant OCH1 gene and expression of mannosidase.

As used herein, the mutant OCH1 gene includes mutations that alter the Golgi localization of the mutant OCH1 protein as compared to the wild type OCH1 protein. For example, the OCH1 protein includes an altered N-terminal region that (i) does not properly target the appended protein to the Golgi apparatus, (ii) not properly retained within the Golgi apparatus, or (iii) not properly targeted or retained within the correct compartment within the Golgi. In some embodiments, the alteration in the N-terminal sequence is a result of a mutation, i.e., addition, deletion or substitution, of one or more amino acids in the membrane anchor domain of the OCH1 protein. In specific embodiments, one or more amino acids in the membrane anchor domain have been deleted. In particular embodiments, at least 2, 3, 4, 5, 6, 7, or more amino acids, contiguous or otherwise, of the membrane anchor domain have been deleted. For example, some or all of the first 5 amino acids (FYMAI, SEQ ID NO: 17) of the membrane anchor domain are deleted. In other embodiments, the alteration in the N-terminal sequence is a result of a mutation, i.e., addition, deletion or substitution, of one or more amino acids in the cytoplasmic tail of the OCH1 protein. In specific embodiments, one or more amino acids in the cytoplasmic tail have been deleted; for example, at least 2, 3, 4, 5, 6, 7, or more amino acids, contiguous or otherwise, of the cytoplasmic tail have been deleted. In other embodiments, deletion of one or more amino acids is combined with addition of one or more amino acids in the cytoplasmic tail. In still other embodiments, the alteration in the N-terminal sequence is a result of a mutation of one or more amino acids in the stem region of the OCH1 protein; for example a deletion of one or more amino acids in the first 10, 20, 30, 40, 50, or 60 amino acids immediately following the membrane anchor domain.

In certain embodiments, the alteration in the N-terminal sequence is a result of a combination of mutations in the cytoplasmic tail, the membrane anchor domain, and/or the stem region of the OCH1 protein. In specific embodiments, the alteration in the N-terminal sequence is a result of a combination of mutations in the cytoplasmic tail and the membrane anchor domain. For 30 example, one or more amino acids in the cytoplasmic tail and one or more amino acids in the membrane anchor domain have been deleted. Examples of deletions in the N-terminal region of the OCH1 protein include deletion of 5, 6, 7, 8, 9, 10, 11, 12, 13, 14, 15, 16, 17, 18, 20, 21, 22, 23, 24, or 25 of the N-terminal amino acids. In other embodiments, in addition to deletions in one or more domains, one or more amino acids are added to the N-terminus of the protein, as long as the resulting mutant N terminal sequence still fails to properly target or localize the OCH1 protein in Golgi. For example, the resulting mutant N-terminal sequence still lacks a functional membrane anchor domain. Whether a mutant sequence includes a membrane anchor domain can be readily determined based on the amino acid compositions and length. The membrane anchor domain of Golgi glycosyltransferases typically consists of 16-20 amino acids, which are hydrophobic and often contain aromatic amino acids, and has hydrophilic, often positively charged amino acids immediately outside both ends of the membrane span. See, e.g., Nakayama et al. (1992), supra. One example of a mutant OCH1 protein is set forth in SEQ ID NO: 4, which has its first 10 amino acids in place of the first 26 amino acids of the wild type OCH1 protein. The mutant OCH1 protein contains a catalytic domain substantially identical to that of the wild type OCH1 protein. The catalytic domain of the wild type OCH1 protein is located within the C-terminal fragment of 360 amino acids (i.e., within amino acids 45 to 404 of SEQ ID NO: 15). In some embodiments, the mutant OCH1 protein includes a C-terminal fragment that is substantially identical to amino acids 45-404, 55-404, 65-404, 75-404, 85-404, 95-404, or 105-404 of SEQ ID NO: 15.

By “substantially identical” it means that the sequences, when aligned in their full lengths, are at least about 90%, 95%, 98%, 99%, or greater, identical. In most embodiments, the catalytic domain of the mutant OCH1 protein does not differ from the wild type of domain by more than 10 amino acids, 8 amino acids, 5 amino acids, 3 amino acids, or 2 amino acids. In specific embodiments, the catalytic domain of the mutant OCH1 protein is identical with that of the wild type OCH1 protein. When one or more amino acids are different, it is preferable that the differences represent conservative amino acid substitutions.

Examples of conservative substitutions include the substitution of a non-polar (hydrophobic) residue such as I, V, L or M for another; the substitution of one polar (hydrophilic) residue for another polar residue, such as R for K, Q for N, G for S, or vice versa; and the substitution of a basic residue such as K, R or H for another or the substitution of one acidic residue such as D or E for another. The mutant OCH1 protein also substantially retains the catalytic activity of the wild type OCH1 protein, i.e., at least about 75%, 80%, 85%, 90%, 95%, or more, of the α-1,6-mannosyltransferase activity of the wild type OCH1 protein. The activity of a particular OCH1 mutant protein can also be readily determined using in vitro or in vivo assays known in the art. See, e.g., Nakayama (1992), supra. As used herein, mannosidase includes α-1,2-Mannosidase and functional equivalents thereof which converts Man8GlcNAc2 to Man5GlcNAc2, thereby providing Man5GlcNAc2 as the predominant N-glycan form. α-1,2-mannosidase (MS-I) is a well characterized family of enzymes. Most MS-I enzymes are known to be localized in the Golgi or endoplasmic reticulum, although a few are secreted and have extracellular activity. See Gonzalez et al., Mol Biol Evolution 17:292-300 (2000). The topology of those enzymes that localize to the ER and the Golgi generally includes a luminal catalytic domain and an N-terminal transmembrane region. See Herscovics, Biochimie 8:757-62 (2001). The N-terminal region is composed of a stem region (closest to the luminal catalytic domain), a transmembrane domain, and a cytoplasmic tail. In the secreted MS-I enzymes, the extra-catalytic transmembrane region is also known as a leader sequence, serving as a signal for secretion of the enzyme. Detailed characterizations of various α-1,2-mannosidases can be found in Becker et al. (European J. Cell Biol 79:986-992 (2000)) which studied the MS-I enzymes from mouse and S. cerevisiae and their catalytic domains; Schneikert and Herscovics (Glycobiology 4:445-450 (1994)) which characterized the catalytic activity of a murine MS-I and its catalytic domain; Gonzalez et al. (J. Biol Chem 274:21375-86 (1999)) which examined the activities and domains of several MS-I enzymes, including two enzymes from C. elegans, a human MS-I and the S. cerevisiae MS-I (from the ER); and Maras et al. (J. Biotechnology 77:255-263 (2000)), which characterizes the T. reesei α-1,2-mannosidase as belonging to the category of secretory MSI's, which are composed of a catalytic domain and an N-terminal leader sequence.

The nucleic acid molecule encoding an α-1,2-mannosidase or a functional fragment thereof can derive from any species for use in this invention, including but not limited to mammalian genes encoding, e.g., murine α-1,2-mannosidase (Herscovics et al. J. Biol. Chem. 269:9864-9871, 1994), rabbit α-1,2-mannosidase (Lal et al. J. Biol. Chem. 269:9872-9881, 1994), or human α-1,2-mannosidase (Tremblay et al. Glycobiology 8:585-595, 1998), fungal genes encoding, e.g., Aspergillus α-1,2-mannosidase (msdS gene), Trichoderma reesei α-1,2-mannosidase (Maras et al., J. Biotechnol. 77:255-263, 2000), or a Saccharomyces cerevisiae α-1,2-mannosidase, as well as other genes such as those from C. elegans (GenBank Accession Nos. CAA98114 and CAB01415) and Drosophila melanogaster (GenBank Accession No. AAF46570) (see, e.g., Nett et al., Yeast 28:237-252, 2011, incorporated herein by reference).

By “functional part” or “enzymatically active fragment” of an α-1,2-mannosidase, it is meant a polypeptide fragment of a naturally occurring or wild type α-1,2-mannosidase which substantially retains the enzymatic activity of the full-length protein. By “substantially” in this context is meant at least about 75%, 80%, 85%, 90%, 95% or more, of the enzymatic activity of the full-length protein is retained. For example, the catalytic domain of an α-1,2-mannosidase, absent of any N-terminal transmembrane or signal sequence, constitutes a “functional fragment” of the α-1,2-mannosidase. Those skilled in the art can readily identify and make functional fragments of an α-1,2-mannosidase based on information available in the art and a combination of techniques known in the art. The activity of a particular polypeptide fragment can also be verified using in vitro or in vivo assays known in art.

In some embodiments, the nucleotide sequence coding for an α-1,2-mannosidase or a functional fragment is derived from the Trichoderma reesei α-1,2-mannosidase coding sequence. In specific embodiments, the nucleotide sequence codes for the Trichoderma reesei α-1,2-mannosidase described by Maras et al. J. Biotechnol. 77:255-63 (2000), or a functional fragment thereof (such as the C-terminal catalytic domain of the full-length protein). In most embodiments, the strains are engineered such that the α-1,2-mannosidase or a functional fragment is targeted to the ER. In specific embodiments, the ER-targeting is achieved by including an ER-targeting sequence in the α-1,2-mannosidase or a functional fragment. Examples of ER-targeting sequences, i.e., sequences that target a protein to the ER so that the protein is localized or retained in the ER, include an N-terminal fragment of S. cerevisiae SEC12, an N-terminal sequence of S. cerevisiae α-glucosidase I encoded by GLS1, and an N-terminal fragment of S. cerevisiae α-1,2-mannosidase encoded by MNS1. See, also, Nett et al. (2011), supra. In a specific embodiment, the α-1,2-mannosidase or a functional fragment is targeted to the ER by including an ER-retention signal, HDEL (SEQ ID NO: 18), at the C-terminal of the α-1,2-mannosidase or its functional fragment.

The nucleic acid coding for an α-1,2-mannosidase or a functional fragment can be introduced through an expression vector into a Pichia pastoris strain. The expression vector can be an integrative vector designed to integrate α-1,2-mannosidase coding sequence into the host genome; or a replicative vector (e.g., a plasmid) which replicates in the strain independent of the chromosomes.

In cases of an integrative vector, the vector can be designed to achieve integration of the nucleic acid into the wild type OCH1 allele (e.g., through single or double cross over homologous recombination) and simultaneous disruption of the wild type OCH1 allele. The mutant OCH1 protein contains a catalytic domain substantially identical to that of the wild type OCH1 protein. In one embodiment, the cell in a cell-based expression system includes an engineered expression vector that includes a mutant OCH1 gene and a mannosidase gene, wherein the engineered expression strain does not contain a WT OCH1 gene. In further embodiments, the expression strains can be additionally modified to express other, downstream enzymes (or functional fragments thereof) in the glycosylation pathway towards making hybrid- and complex-type N-glycans. Such additional enzymes include, e.g., one or more of GlcNAc transferase I (GnT-I), β-1,4-galactosyltransferase 1 (GalT), mannosidase II (Man-II), and GnT-II, among others. See Jacobs et al. (2009); U.S. Pat. No. 7,029,872 to Gerngross. GnT-I catalyzes the addition of a β-1,2-linked GlcNAc residue to the α-1,3-mannose 25 of the trimannosyl core in Man5GlcNAc2.

Introduction of the GnT-I activity can be achieved by transforming with a vector including a nucleic acid sequence coding for a GlcNActransferase I (GnT-I) for use in this invention. Such nucleic acid sequence can derive from any species, e.g., rabbit, rat, human, plants, insects, nematodes and protozoa such as Leishmania tarentolae. In specific embodiments, the nucleotide sequence encodes a human 30 GnT-I. The GnT-I or a functional part thereof is targeted to the Golgi apparatus, which can be achieved by including a yeast Golgi localization signal in the GnT-I protein or a functional part thereof. In certain embodiments, the catalytic domain of human GnT-I is fused to the N-terminal domain of S. cerevisiae Kre2p, a glycosyltransferase with a known cis/medial Golgi localization. GalT catalyzes the addition of a galactose residue in β-1,4-linkage to the β-1,2-5 GlcNAc, using UDP-Gal as donor substrate. Introduction of the GalT activity can be achieved by transforming with a vector including a nucleic acid sequence coding for a GalT or a functional fragment thereof, which can derive from human, plants (e.g. Arabidopsis thaliana), insects (e.g. Drosophila melanogaster). The GalT or a functional part thereof is genetically engineered to contain a Golgi-retention signal and is targeted to the Golgi apparatus.

An exemplary Golgi-retention signal is composed of the first 100 amino acids of the Saccharomyces cerevisiae Kre2 protein. Man-II acts to remove both terminal α-1,3- and α-1,6-mannoses from GlcNAcMan5GlcNAc2 N-glycans. The presence of a terminal-1,2-linked GlcNAc residue on the α-1,3-arm is essential for this activity. Introduction of the Man-II activity can be achieved by transforming a strain with a nucleic acid vector coding for a Man-II protein or a functional fragment thereof, engineered to contain a Golgi-localization signal. As an example, a suitable nucleic acid can encode the catalytic domain of Drosophila melanogaster Man-II, fused in frame to the Golgi-localization domain of S. cerevisiae Mnn2p. GnT-II catalyzes the addition of a second β-1,2-linked GlcNAc residue to the free α 1,6-mannose of the trimannosyl core.

Introduction of the GnT-II activity can be achieved by transforming with a vector which contains a nucleotide sequence coding for a GnT-II protein or a functional fragment thereof. GnT-II genes have been cloned from a number of species including mammalian species and can be used in the present invention. As an example, a suitable nucleotide sequence codes for the catalytic domain of rat GnT-II fused to the N terminal part of S. cerevisiae Mnn2p.

The strains disclose herein can include additional features, achieved by various suitable manipulations (such as cross or recombinant engineering), including, e.g., having a mutant auxotroph gene (e.g., his-) to facilitate cloning and selection, having protease deficiency for limiting product degradation (e.g., pep4-, prbl-, and/or sub2-), having a slow methanol utilization phenotype (e.g., mutS). In specific embodiments, following strains can be used: SuperMan5, P. pastoris, och1-, blasticidin resistant, Mannosidase I from T. reesei (=His+); SuperMan5 (his-), P. pastoris, och1-, his4-, blasticidin resistant, Mannosidase I from T. reesei; SuperMan5 (mutS), P. pastoris, och1-, blasticidin resistant, Mannosidase I from T. reesei (slow methanol utilization); SuperMan5 (pep4-), P. pastoris, och1-, blasticidin resistant, Mannosidase I from T. reesei (protease deficient); SuperMan5 (prbl-), P. pastoris, och1-, blasticidin resistant, Mannosidase I from T. reesei (protease deficient); SuperMan5 (pep4-, sub2-), P. pastoris, och1-, blasticidin resistant, Mannosidase I from T. reesei (protease deficient); and SuperMan5 (pep4-, prbl-), P. pastoris, och1-, blasticidin resistant, Mannosidase I from T. reesei (protease deficient). In one embodiment, the cell-based system includes the Pichia GlycoSwitch® systems, which are described in patent publications US20150267212 and WO/2015/100058. Construction and expression of human alpha-1 anti-trypsin (A1AT) in new Pichia pastoris GlycoSwitch® strain, Man5 N-linked oligosaccharide structures designated as SuperMan5 expression strain with an ochl mutation, and the addition of the mannosidase I gene. The SuperMan5 expression strain (HIS4+, Och1-disruption with a pGAP-mannosidase expression cassette, blasticidin resistant) GS115 with the mutation at the HIS4 gene reverted to wild type (HIS4+). The alpha 1,2-mannosidase from T. reesei regulated by the GAP promoter on a plasmid with the Blasticidin resistance gene disrupting the Och1 gene in the SuperMan5 genome. This strain is also described in Jacobs et al. (2009), Nature Protocols 4:58-70 (incorporated herein by reference).

The protein or polypeptide of the invention can also be synthesized in cell-free systems, using, for example, cell extracts or ribosomes. Modified or mutant proteins can be added to or used in the cell-free system. Such proteins include those that provide post translational modifications, such as glycosylation. For example, the cell-free system can include mutant OCH1 protein and a mannosidase protein. Advantageously, the protein or polypeptide can be produced as a fusion with a second protein, such as Glutathione-S-transferase (GST) or the like, or a sequence tag, such as the Histidine tag (His-tag) sequence.

The use of fusion or tagged proteins simplifies the purification procedure, as detailed in the above-noted Current Protocols in MolecularBiology, and in the instructions for His-tag protein expression and purification kits (available, e.g. from Qiagen GmbH, Germany). In one embodiment, the present invention provides a recombinant DNA molecule that encodes for the rALAT protein disclosed herein. In one embodiment, recombinant DNA encoding for recombinant alpha-1 antitrypsin protein described above is expressed in a suitable expression vector under the control of an inducible promoter.

Compositions as Therapeutics in certain embodiments the presently disclosed recombinant rA1AT glycoprotein compositions can be administered in combination with one or more additional compounds or agents (“additional active agents”) for the treatment, management, and/or prevention of, among other things, A1AT mediated lung or topical diseases or disorders including but not limited to treatment for inflammatory and immune disorders, such as, but not limited to, autoimmune diseases. Such therapies can be administered to a patient at therapeutically effective doses to treat or ameliorate, among other things, to modulate production of pro- and anti-inflammatory molecules. A therapeutically effective dose refers to that amount of the compound sufficient to result in any delay in onset, amelioration, or retardation of disease symptoms. Additionally, the bioactive agent can be coupled or complexed with a variety of well-established compositions or structures that, for instance, enhance the stability of the bioactive agent, or otherwise enhance its pharmacological properties (e.g., increase in vivo half-life, reduce toxicity, etc.).

Pharmaceutical compositions for use in accordance with the presently described compositions can be formulated in conventional manners using one or more physiologically acceptable carriers or excipients. The details are set forth in the aforesaid parent application, which has been incorporated herein by reference in its entirety.

### Method of Use/Treatment of A1AT Mediated Processes

In one embodiment, the present invention provides a method of treating A1AT mediated lung or topical disease or disorder. The effectiveness of commercial preparations of A1AT (like Prolastin®, Aralast®, or Zameira®) is based on elastase inhibitory activity. Therapy with weekly intravenous infusions of Prolastin® was introduced 30 years ago to treat emphysema patients with inherited A1AT deficiency (A1ATD).

The concept behind this therapy is to protect lung tissues from unregulated proteolysis and to slow down progression of emphysema. To date, clinical studies show only modest benefit, although in most cases therapy is well tolerated. To achieve therapeutic effect, high levels of Prolastin® are needed (typically 60 mg per kg body weight weekly). Preparations of clinical grade A1AT are expensive, and their availability is limited; functional properties of the A1AT protein are affected by the complicated methods of purification and by pasteurization processes of human plasma products. The A1AT described herein can be used in the treatment of A1AT mediated lung or topical disease or disorder.

Examples of A1AT mediated lung disease or disorder includes emphysema, asthma, pulmonary disease, and chronic obstructive pulmonary disease (COPD). The A1AT described herein can be used in the treatment of A1AT mediated topical disease or disorder. Examples of A1AT mediated topical disease or disorder includes eye and ear otitis media otitis externa. arthritis, conjunctivitis, hot spots, atopic dermatitis, skin wound, pruritus, skin inflammation, psoriasis, and mast cell tumors. As used herein “skin wound” includes any disruption of the skin, including abrasions, avulsions, cuts, lacerations, and punctures. In one embodiment, the A1AT described herein is used in the treatment of eye and ear otitis media otitis externa, arthritis, conjunctivitis, hot spots, atopic dermatitis, skin wound, pruritus, skin inflammation, psoriasis, and mast cell tumors in a non-human animal.

The presently disclosed rA1AT has been determined to have increased bioactivity, as described herein, as compared to isolated and purified natural A1AT protein. Therefore, disease and disorders that are known or suspected of benefiting from treatment purified human A1AT will also benefit from treatment using rA1AT, with the added benefits of reduced risk of transmitting infectious agents, reduced cost of production and reduced dosage requirements due to increased bioactivity. Therapy with A1AT became accepted based on biochemical efficacy. Most of the clinical efficacy studies of A1AT therapy had relied on nonrandomized prospective observational approaches that, although underpowered, included the changes in lung function decline as a main outcome. According to findings of RAPID (Randomized, Placebo controlled Trial of Augmentation Therapy in Alpha-1 Proteinase Inhibitor Deficiency) study, published in The Lancet, 2015, patients treated with A1AT therapy exhibited a lower annual rate of lung density decline compared to placebo, when measured using chest computed tomography. The large number of disorders for which A1AT has been proposed as a therapeutic agent, coupled with the general disadvantages of agents derived from pooled human plasma, including the presence of copurifying protein contaminants and the risk of transmitting infectious agents, has motivated the development of recombinant A1AT and engineered A1AT muteins. Recombinant A1AT has been produced, for example, in yeast, Rosenberg et al., Nature 312:77-80 (1984) (rA1AT); and in plants, Terashima et al., Appl. Microbiol. Biotechnol. 52:516-23 (1999) and Huang et al., Biotechnol. Prog. 17:126-33 (2001).

In addition to the indications for which A1AT therapy have been approved, there are additional indications for which treatment with A1AT or preferably rA1AT has been indicated, these examples of the effects of therapy with A1AT (validated in mouse models of human disease) include but are not limited to following examples. Described herein is a recombinant A1AT (rA1AT), which has excellent immunomodulatory properties that indicate it is a highly active biotherapeutic, for example in ex vivo models, rA1AT was effective at 20 times lower doses than Prolastin®, it was stable and it is easy to synthesize, will be less expensive and rA1AT promises an immunomodulatory therapy that can be used to prevent or treat a wide range of diseases and disorders. Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions which exhibit large therapeutic indices are preferred.

While compositions that exhibit toxic side effects can be used, care should be taken to design a delivery system that targets such compositions to the site of affected tissue in order to minimize potential damage to uninfected cells and, thereby, reduce side effects. The data obtained from cell culture assays and animal studies can be used in formulating a range of dosage for use in humans. The dosage of such compositions lies preferably within a range of circulating concentrations that include the ED50 with little or no toxicity. The dosage can vary within this range depending upon the dosage form employed and the route of administration utilized. For any compositions used in the method of the invention, the therapeutically effective dose can be estimated initially from cell culture assays. A dose can be also formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, ELISA or RIA. A therapeutically effective dose refers to that amount of the composition sufficient to result in any amelioration or retardation of disease symptoms or progression.

When the therapeutic treatment of disease is contemplated, the appropriate dosage can also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including humans. Before human studies of efficacy are undertaken, Phase I clinical studies in normal subject's help establish safe doses. Additionally, the bioactive compositions can be complexed with a variety of wellestablished compounds or structures that, for instance, enhance the stability of the bioactive agent, or otherwise enhance its pharmacological properties (e.g., increase in vivo half-life, reduce toxicity, etc.). The above therapeutic compositions can be administered as described throughout this specification by any number of methods known to those of ordinary skill in the art including, but not limited to, administration by inhalation; by subcutaneous (sub-q), intravenous (I.V.), intraperitoneal (I.P.), intramuscular (I.M.), or intrathecal injection; or as a topically applied agent (transdermal (patch), ointments, creams, salves, eye drops, and the like).

The therapeutic compositions can be administered via implantation, in a controlled released fashion. Generally, in humans, oral administration of a pharmaceutical composition including rA1AT glycoprotein, according to the invention, can be administered alone but will generally be administered in admixture with a suitable pharmaceutical excipient, diluent or carrier selected with regard to the intended route of administration and standard pharmaceutical practice. Specific methods by which the rA1AT glycoprotein composition, a pharmaceutically acceptable salt, and pharmaceutically acceptable solvates of either entity, when used in accordance with the invention, can be administered for human medical use include oral administration by capsule, bolus, tablet and many methods are known to enhance oral delivery of bioactive peptides, such as rA1AT, including, but not limited to co-administration with bile salts (sodium deoxycholate, sodium taurocholate, sodium glycodeoxycholate, sodium taurodihydrofusidate, sodium glycodihydrofudisate), chelators (EDTA, citric acid, salicylates), surfactants (sodium lauryl sulfate, laureth-9, sodium dodecylsulfate, sodium taurodihydrofusidate, poly oxyethylene ethers), fatty acids and derivatives (oleic acid, linoleic acid, caprylic acid, capric acid, acyl carnitines, mono and di-glycerides), cationic polymers (chitosan and its derivatives), anionic polymers (carbopol and polyacrylic acid derivatives, N-acetyl cysteine, acylcarnitine) and acylcarnitines (Lauroyl-L-carnitine chloride, palmitoylcarnitine chloride).

One embodiment of an oral dosing regimen is from 10 μg and 500 mg of composition when required. In circumstances where the recipient suffers from a swallowing disorder or from impairment of drug absorption after oral administration, the drug can be administered parenterally, sublingually or buccally in the form of tablets, capsules (including soft gel capsules), ovules, elixirs, solutions or suspensions, which can contain flavoring or coloring agents, for immediate-, delayed-, modified-, or controlled-release such as sustained-, dual-, or pulsatile delivery applications. The rALAT glycoprotein, and pharmaceutically acceptable solvates of either entity, when used in accordance with the invention, can also be administered via fast dispersing or fast dissolving dosages forms or in the form of a high energy dispersion or as coated particles. Suitable pharmaceutical formulations of the rALAT glycoprotein, pharmaceutically acceptable salt, and pharmaceutically acceptable solvates of either entity, when used in accordance with the invention, can be in coated or uncoated form as desired.

In general, a tablet formulation could typically contain between about 10 μg and 500 mg of a composition such as rALAT glycoprotein, or a pharmaceutically acceptable salt, or pharmaceutically acceptable solvate of either entity, when used in accordance with the invention, whilst tablet fill weights often can range from 50 mg to 1000 mg. An example formulation for a 10 mg tablet is illustrated by the following example that is intended to be illustrative only and not intended to limit the scope of the invention. Free acid, free base or salt of composition (quantity adjusted according to bioactivity), 10.000% w/w; lactose, 64.125% w/w; starch, 21.375% w/w; croscarmellose sodium, 3.000% w/w; and magnesium stearate, 1.500% w/w. The tablets are manufactured by a standard process, for example, direct compression or a wet or dry granulation process. Tablet cores can be coated with appropriate overcoats as desired. An additional example for illustration only and are not intended to limit the scope of the invention. Active ingredient means a rALAT glycoprotein composition, or a pharmaceutically acceptable salt of the invention.

A tablet is prepared using the following ingredients: active ingredient, 250 mg; cellulose, microcrystalline, 400 mg; silicon dioxide, fumed, 10 mg; and stearic acid, 5 mg for a total of 665 mg. The components are blended and compressed to form tablets each weighing 665 mg. Solid compositions of a similar type can also be employed as fillers in gelatin capsules. Preferred excipients in this regard include lactose, starch, cellulose, milk sugar or high molecular weight polyethylene glycols.

For aqueous suspensions and/or elixirs, the compounds of the invention can be combined with various sweetening or flavoring agents, coloring matter or dyes, with emulsifying and/or suspending agents and with diluents such as water, ethanol, propylene glycol and glycerin, and combinations thereof. Modified release and pulsatile release dosage forms can contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms can contain one or a combination of release rate modifying excipients. Release rate modifying excipients can be present both within the dosage form, i.e., within the matrix, and/or on the dosage form, i.e., upon the surface or coating. Fast dispersing or dissolving dosage formulations (FDDFs) can contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavoring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used, i.e., where the drug substance is insoluble, a fast-dispersing dosage form can be prepared and where the drug substance is soluble, a fast dissolving dosage form can be prepared. Additional embodiments of the present invention include rALAT glycoprotein compositions that demonstrate activity and selectivity in vitro as determined, for example, using the assays described in the present application.

In certain embodiments, desirable modulators will also have some or all of the following properties. rA1AT glycoprotein compositions should preferably be soluble at greater than 0.1 μM per ml, more preferably greater than 1 μM per ml and more preferably still at 10 μM per ml. The rA1AT glycoprotein compositions should have no effect in a cytotoxicity assay at the desired dose and preferably at 10 times the desired dose and more preferably at 100 times the desired dose. rA1AT glycoprotein compositions that demonstrate a linear PK dose-dependency and bioavailability (for example greater than 25% unbound concentrations (Cp) or tissue concentrations (Ctissue) greater than 5-10×IC50 with qid or bid dosing) in a number of species (mice, rats, dogs, non-human primates, humans) at therapeutic concentrations might be preferable.

Additionally, rA1AT glycoprotein compositions that have a metabolic stability of greater than 60% and preferably greater than 80%, might be desired. In additional embodiments, rA1AT glycoprotein compositions should also demonstrate a therapeutic index greater than 1 and preferably greater than 10 or greater than 50 or 100. rA1AT glycoprotein compositions would also have a bioavailability of greater than 25% and a half-life (T1/2) that is greater than 2-4 hours, or greater than 4-6, or 6-8 hours, or 8-10 hours, or 10-12 hours, or 12-14 hours, or 14-16 hours, or 16-18 hours, or 18-20 hours, or 20-22 hours, or 22-24 hours, or 24-48 hours, or 48-60 hours, or longer. Desirable rALAT glycoprotein compositions should also fail to demonstrate cytochrome P450 (CYP) 3A4, 2D6, 1A2, or 2C9 inhibition of greater than 3 μM or greater than 1000 times target selectivity if the CYP IC50 is less than 3 μM, with a less than 25-30% contribution by those CYPs with significant polymorphisms (i.e., 2D6 and 2C19).

The terms “pharmaceutical” and “pharmaceutically acceptable” as used herein are also in relation to excipients, diluents, carriers, salts, solvates, etc. The rA1AT glycoprotein compositions, pharmaceutically acceptable salts, and pharmaceutically acceptable solvates of either entity, when used in accordance with the invention, can also be administered parenterally, for example, intravenously, intra-arterially, intraperitoneally, intrathecally, intraventricularly, intraurethrally, intrasternally, intracranially, intramuscularly or subcutaneously, or they can be administered by infusion or needleless injection techniques. For such parenteral administration they are best used in the form of a sterile aqueous solution that can contain other substances, for example, enough salts or glucose to make the solution isotonic with blood. The aqueous solutions should be suitably buffered (a pH of from 3 to 9), if necessary. The preparation of suitable parenteral formulations under sterile conditions is readily accomplished by standard pharmaceutical techniques well known to those skilled in the art. Sterile and pyrogen free purified rA1AT glycoprotein compositions can be used to treat humans suffering from symptoms associated with A1AT related diseases and disorders, such as, but not limited to immune or inflammatory disorders. Purified rA1AT glycoprotein preparations are applied by intravenous infusion over time in the presence of a physiologically acceptable solvent (saline, dextrose solution, etc.) or by bolus injection (subcutaneous, intramuscular, or intraperitoneal). The rA1AT glycoprotein compositions, pharmaceutically acceptable salts, and pharmaceutically acceptable solvates of either entity, when used in accordance with the invention, can also be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A) or 1,1,1,2,3,3,3-heptafluoropropane (HFA 227EA), carbon dioxide or other suitable gas.

In the case of a pressurized aerosol, the dosage unit can be determined by providing a valve to deliver a metered amount. The pressurized container, pump, spray or nebulizer can contain a solution or suspension of the active composition, e.g. using a mixture of ethanol and the propellant as the solvent, which can additionally contain a lubricant, e.g. sorbitan trioleate. Capsules and cartridges (made, for example, from gelatin) for use in an inhaler or insufflator can be formulated to contain a powder mix of a compound of the invention and a suitable powder base such as lactose or starch. Aerosol or dry powder formulations are arranged so that each metered dose or “puff” contains from 1 μg to 50 mg of a rALAT glycoprotein compositions, pharmaceutically acceptable salts, or pharmaceutically acceptable solvate of either entity, when used in accordance with the invention, for delivery to the patient. The overall daily dose with an aerosol will be in the range of from 1 μg to 50 mg that can be administered in a single dose or, more usually, in divided doses throughout the day.

The rA1AT glycoprotein, pharmaceutically acceptable salts, and pharmaceutically acceptable solvates of either entity, when used in accordance with the invention, can also be formulated for delivery via an atomizer. Formulations for atomizer devices can contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, and oleic acid. Alternatively, the rA1AT glycoprotein compositions, pharmaceutically acceptable salt, and pharmaceutically acceptable solvates of either entity, when used in accordance with the invention, can be administered in the form of a suppository or pessary, or they can be applied topically in the form of a gel, hydrogel, lotion, solution, cream, ointment or dusting powder.

The rALAT glycoprotein compositions, pharmaceutically acceptable salts, and pharmaceutically acceptable solvates of either entity, when used in accordance with the invention, can also be dermally administered. The rA1AT glycoprotein compositions, pharmaceutically acceptable salts, and pharmaceutically acceptable solvate of either entity, when used in accordance with the invention, can also be transdermally administered, for example, by the use of a skin patch. They can also be administered by the ocular, pulmonary or rectal routes. For ophthalmic use, the rA1AT glycoprotein compositions, pharmaceutically acceptable salts, and pharmaceutically acceptable solvates of either entity, when used in accordance with the invention, can be formulated as micronised suspensions in isotonic, pH adjusted, sterile saline, or, as solutions in isotonic, pH adjusted, sterile saline, optionally in combination with a preservative such as a benzylalkonium chloride. Alternatively, they can be formulated in an ointment such as petrolatum. For application topically to the skin, the rA1AT glycoprotein compositions, pharmaceutically acceptable salts, and pharmaceutically acceptable solvates of either entity, when used in accordance with the invention, can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyidodecanol, benzyl alcohol and water. To enhance the delivery of rA1AT glycoprotein composition-based preparations, peptide carriers or delivery systems such as microspheres, nanoparticles, PEG and polyvinylpyrrolidone (PVP) polymers, along with lipid-based systems can be used. The addition of lipidic (e.g. lipoamino acids) and/or hydrophilic units (e.g. carbohydrates, PEG) to potential peptide therapeutics provides an effective strategy for enhanced drug delivery in vivo.

Similarly, such rALAT glycoprotein compositions can be chemically modified using acylation, lipidation or PEGylation, to increase resistance to proteases and rapid renal clearance and thus extending half-life and increase the periods between dosing. Polymers are also used to enhance the delivery of peptides, either by attachment or encapsulation. Another method of improving the stability of rA1AT glycoprotein preparations is to substitute unnatural (L for D) amino acids, or cyclization of the peptide sequence. Peptidomimetics, modifications to peptide bonds have improved peptide stability. While bioadhesion can be enhanced by co-administering TA1AT glycoprotein in combination with chitosans or mucoadhesive systems. Inclusion of rA1AT glycoprotein preparations in hydrogels, three-dimensional mesh including hydrophilic polymers that incorporate large amounts of water and form a gel like matrix due to cross-linking can provide an efficient and convenient way to administer such peptides and proteins.

Effective therapeutic compositions of rA1AT glycoprotein can also include incorporation alone or in combination with other active agents, into liposomes, and microparticles or nanoparticles (nanospheres and nanocapsules) some including polyethylene glycol (PEG) ylated polylactide-co-glycolide (PLGA) or a multiarm PEG polymer, such as Nacetyl cysteine linked to a dendrimer, such as a PAMAM dendrimer (as described, for example in US20120003155). The TALAT glycoprotein composition when used in accordance with the invention, can also be used in combination with cyclodextrin. Cyclodextrins are known to form inclusion and non-inclusion complexes with drug molecules. The formation of a drug cyclodextrin complex can modify the solubility, dissolution rate, bioavailability, and/or stability property of a composition.

Cyclodextrin complexes are generally useful for most dosage forms and administration routes. As an alternative to direct complexation with the preparation, cyclodextrin can be used as an auxiliary additive, e.g. as a carrier, diluent or solubilizer. Alpha-, beta- and gamma-cyclodextrins are most commonly used.

Regardless of the manner of administration, the specific dose can be calculated according to established factors such as body weight or body surface and/or based on findings in drug metabolism and pharmacokinetic (DMPK) analyses. Further refinement of the calculations necessary to determine the appropriate dosage for modulating A1AT mediated lung or topical disorders can readily be made by those of ordinary skill in the art without undue experimentation. Methods of determining the level of rALAT circulating in patients include but are not limited to HPLC, immunoassays such as but not limited to ELISA or RIA, and similar well-known methods. Similarly, during the course of treatment, the effects of the rA1AT glycoprotein pharmaceutical compositions can be determined using biochemical markers that relate to the specific A1AT related disease or disorder and the symptom associated with it can be easily obtained bodily fluids, such as urine or serum. These assays and others deemed appropriate based on symptoms, intuition or the results of other medical laboratory techniques, such as radiologic interrogation, are well known to those of skill in the art and are readily available at most medical facilities and hospitals.

### Definitions

As used herein, the terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can be included in a protein's or peptide's sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, and fusion proteins, among others. The polypeptides include natural peptides, recombinant peptides, synthetic peptides, or a combination thereof. In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components including one unit and elements or components that include more than one unit unless specifically stated otherwise. The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls. As used herein, unless otherwise indicated, the term A1AT mediated lung or topical diseases or disorder include those diseases and disorders which can be treated by the application of A1AT, such conditions include, but are not limited to, chronic obstructive pulmonary disease (COPD), asthma, bronchiectasis, emphysema, eye and ear otitis media otitis externa, arthritis, conjunctivitis, hot spots, atopic dermatitis, skin wound, pruritus, skin inflammation, mast cell tumors, and modulation of the production of pro- and anti-inflammatory molecules and thus regulating the inflammatory response and down-regulating hyper-immunity or hyper-inflammation in A1AT mediated lung or topical diseases or disorder. As used herein, and unless otherwise indicated, the terms “treat,” “treating,” “treatment” and “therapy” contemplate an action that occurs while a patient is suffering from an A1AT mediated lung or topical disease or disorder that reduces the severity of one or more symptoms or effects of an A1AT mediated lung or topical disease or disorder. Where the context allows, the terms “treat,” “treating,” and “treatment” also refers to actions taken toward ensuring that individuals at increased risk of an A1AT mediated lung or topical disease or disorder, are able to receive appropriate surgical and/or other medical intervention prior to onset of an A1AT mediated lung or topical disease or disorder. As used herein, and unless otherwise indicated, the terms “prevent,” “preventing,” and “prevention” contemplate an action that occurs before a patient begins to suffer from an A1AT mediated lung or topical disease or disorder, that delays the onset of, and/or inhibits or reduces the severity of an A1AT mediated lung or topical disease or disorder. As used herein, and unless otherwise indicated, the terms “manage,” “managing,” and “management” encompass preventing, delaying, or reducing the severity of a recurrence of an A1AT mediated lung or topical disease or disorder in a patient who has already suffered from such a disease, disorder or condition. The terms encompass modulating the threshold, development, and/or duration of the A1AT mediated lung or topical disease or disorder or changing how a patient responds to an A1AT mediated lung or topical disease or disorder. As used herein, and unless otherwise specified, a “therapeutically effective amount” of a compound is an amount sufficient to provide any therapeutic benefit in the treatment or management of an A1AT mediated lung or topical disease or disorder or to delay or minimize one or more symptoms associated with an A1AT mediated lung or topical disease or disorder. A therapeutically effective amount of a compound means an amount of the compound, alone or in combination with one or more other therapies and/or therapeutic agents that provide any therapeutic benefit in the treatment or management of an A1AT mediated lung or topical disease or disorder. The term “therapeutically effective amount” can encompass an amount that alleviates an A1AT mediated lung or topical disease or disorder, improves or reduces an A1AT mediated lung or topical disease or disorder, improves overall therapy, or enhances the therapeutic efficacy of another therapeutic agent. As used herein, and unless otherwise specified, a “prophylactically effective amount” of a compound is an amount sufficient to prevent or delay the onset of an A1AT mediated lung or topical disease or disorder, or one or more symptoms associated with an A1AT mediated lung or topical disease or disorder or prevent or delay its recurrence. A prophylactically effective amount of a compound means an amount of the compound, alone or in combination with one or more other treatment and/or prophylactic agent that provides a prophylactic benefit in the prevention of an A1AT mediated lung or topical disease or disorder. The term “prophylactically effective amount” can encompass an amount that prevents an A1AT mediated lung or topical disease or disorder, improves overall prophylaxis, or enhances the prophylactic efficacy of another prophylactic agent. The “prophylactically effective amount” can be prescribed prior to, for example, the development of an A1AT mediated lung or topical disease or disorder. As used herein, “patient” or “subject” includes mammalian organisms which are capable of suffering from an A1AT mediated lung or topical disease or disorder as described herein, such as human and non-human mammals. Human patients can include adults, juveniles and infants. In one embodiment, a “patient” or “subject” includes non-human mammals. Examples of non-human mammals include pets and farm or food animals. Examples of pets include dogs and cats. Examples of farm or food animals include cows, rabbits, sheep, goats, chickens, horses, and pigs. As used herein, the terms “biological activity”, “bioactivity” and “biologically active human A1AT” or “A1AT bioactivity” refer to any biological activity associated with native A1AT, or any isolated and purified native A1AT proteins. Increased bioactivity is a bioactivity level which is greater than the levels of bioactivity that would be demonstrated by isolated and purified native A1AT proteins. Decreased bioactivity is a bioactivity level which is less than the levels of bioactivity that would be demonstrated by isolated and purified native A1AT proteins. Reduce or limit “limit” the damage caused by activated inflammatory response, such as but not limited to the inhibition of increases in inflammation associated cytokines, for example, IL-1, IL-6, and TNF alpha, as well as LPS-induced monocyte chemoattractant protein-1 (MCP-1), IL-1, and matrixmetalloprotease-9 (MMP9) levels. As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions can be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. As used herein, the term operative linkage or operably associated refers to the relationship among elements of a DNA construct in which the elements are arranged whereby regulatory sequences of nucleotides that are part of the construct directly or indirectly control expression of the DNA in the construct, including DNA encoding a protein or a peptide. As used herein, the term “a DNA fragment operably encoding A1AT peptides” includes DNA fragments encoding A1AT or any other “A1AT peptide” as defined hereinabove. DNA encoding A1AT is known in the art and can be obtained by chemical synthesis or by transcription of messenger RNA (mRNA) corresponding to A1AT into complementary DNA (cDNA) and converting the latter into a double stranded cDNA. Chemical synthesis of a gene for human A1AT has also been contemplated. The requisite DNA sequence can also be removed, for example, by restriction of enzyme digestion of known vectors harboring the A1AT gene. Examples of such vectors and the means for their preparation are well known to those of skill in the art. See, e.g., Niwa et al. (1986) Annals of the NY Academy of Science, 469:31-52, and Buell et al. (1985) Nucleic Acids Research, 13:1923-1938. As used herein, the term expression vector is intended to include vectors capable of expressing DNA that are in operational association with other sequences capable of effecting their expression, such as promoter sequences, in a selected host cell. In general, expression vectors usually used in recombinant DNA technology are often in the form of “plasmids” which are circular, double-stranded DNA loops, extrachromosomal elements. As used herein, the term “culture” means a propagation of cells in a medium conducive to their growth, and all subcultures thereof. The term “subculture” refers to a culture of cells grown from cells of another culture (source culture), or any subculture of the source culture, regardless of the number of times subculturing has been performed between the subculture of interest and the source culture. Further, unless expressly stated to the contrary, “or” refers to an inclusive “or” and not to an exclusive “or”. For example, a condition A or B is satisfied by any one of the following: A is true (or present), and B is false (or not present), A is false (or not present) and B is true (or present), and both A and B are true (or present). Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member can be combined with any one or more of the other members to make additional sub-groups. For example, if the members of a group are a, b, c, d, and e, additional sub-groups specifically contemplated include anyone, two, three, or four of the members, e.g., a and c; a, d, and e; b, c, d, and e; etc. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims. All cited publications, patents, and patent applications are herein incorporated by reference in their entirety.

### Examples

Although the exemplary embodiments detailed below emphasize the use of particular methods of expression, purification and bioactivity characterization, those of skill in the art would readily recognize that these are only exemplary and may not be limiting, as similar compositions can be obtained and characterized using methods mother than those described. Many of the general methodologies described herein are known to the field and can be found in, among others, MOLECULAR CLONING: A LABORATORY MANUAL, Fourth Edition, M. R. Green J. Sambrook (2012) ISBN 978-1-936113-42-2 and “Engineering Complex-Type N-Glycosylation In Pichia Pastoris Using Glycoswitch Technology”, Jacobs PP, Geysens S, Vervecken W, Contreras R, Callewaert N., Nature Protocols. 2009; 4 (1): 58-70, both of which are herein incorporated by reference in their entirety).

### Example 1 Expression Vectors

Construction and expression of human alpha-anti-trypsin (rA1AT) in new Pichia GlycoSwitch® SuperMan5 strains expressing human alpha-antitrypsin (rA1AT) (BioGrammatics) was done after expression in previous strains demonstrated significantly lower rA1AT expression levels. Pichia GlycoSwitch® strains are described in patents publications US20150267212 and WO/2015/100058. Construction and expression of human alpha-anti-trypsin (A1AT) in new Pichia pastoris GlycoSwitch® strain, Man5 N-linked oligosaccharide structures designated as SuperMan5 expression strain with an och1 mutation, and the addition of the mannosidase I gene. SuperMan5, (HIS4+, Och1-disruption with a pGAP-mannosidase expression cassette, blasticidin resistant) GS115 with the mutation at the HIS4 gene reverted to wild type (HIS4+). The alpha 1, 2-mannosidase from T. reesei regulated by the GAP promoter on a plasmid with the Blasticidin resistance gene disrupting the Och1 gene in the SuperMan5 genome.

All expression vectors tested were designed to produce a secreted recombinant human rA1AT protein having the same amino acid sequence. The Sequence Listing shows the amino acid sequences of an A1AT ORF sequence that was designated as hA1AT and was sub cloned from a pPICZalphaA-A1AT expression vector created at Invitrogen (SEQ ID NO: 6). The predicted pPICZalphaA-A1AT vector sequence (SEQ ID NO: 7) that was synthesized as 2 gBlocks (Integrated DNA Technologies, Iowa, USA) shown in SEQ ID NO: 8 and SEQ ID NO: 9. A pPICZalphaA-A1AT expression vector (SEQ ID NO: 7) created at Invitrogen. The predicted pPICZalphaA-A1AT vector sequence follows, with the sequence used in the new expression vector is highlighted in green. This sequence was synthesized as 2 gBlocks (Integrated DNA Technologies, Inc. (SEQ ID NO: 8 and 9) and sub-cloned into both pJAGint and pJANint BioGrammatics vectors, it should be noted that the sub-cloned sequence also codes for the alpha Mating Factor (aMF), as well as, 3′ untranslated sequences that were added to the BioGrammatics vectors. Sequence verification of the entire ORF and cloning junctions was performed prior to transformation (GENEWIZ, LLC: South Plainfield, N.J., USA). However, to optimize expression of a recombinant human ATT, three different A1AT expression vectors were tested, each with a different open reading frame (ORF). While each expression vector had a different DNA sequence contained within the A1AT open reading frame, all sequences were believed to encode the same human A1AT amino acid sequence. The first ORF sequence (SEQ ID NO: 6) was designated as hA1AT and was sub cloned from a pPICZalphaA-A1AT expression vector created at Invitrogen. The predicted pPICZalphaA-A1AT vector sequence (SEQ ID NO: 7) was synthesized as 2 gBlocks (Integrated DNA Technologies, Iowa, USA) shown in SEQ ID NO: 8 and SEQ ID NO: 9) and sub-cloned into both pJAGint and pJANint, it should be noted that the sub-cloned sequence also codes for the alpha Mating Factor (aMF), as well as, 3′ untranslated sequences that were added to the vectors by BioGrammatics. Sequence verification of the entire ORF and cloning junctions was performed prior to transformation (GENEWIZ, LLC). The second ORF sequence (SEQ ID NO: 7) was designated as 2A1AT and contained an A1AT ORF codon that had been optimized at DNA 2.0, Inc. (Menlo Park, CA, USA) for intracellular expression and high-level expression in Pichia.

The third ORF sequence (SEQ ID NO: 11) was designated as 2tA1AT and contained an A1AT ORF codon that had been optimized at DNA 2.0, Inc., for intracellular expression and contained an added the N-term HisTag (8 histidine codons) to facilitate isolation of the recombinant peptide. Each of these three ORFs (hA1AT, 2A1AT, and 2tA1AT) were sub-cloned into the expression vectors pJAG and pJAN and the entire ORF sequences, non-coding sequences and cloning junction sequences of the added DNA were confirmed (GENEWIZ, LLC) as 2 gBlocks (Integrated DNA Techologies, Iowa, USA) DNA, sequences follow) and subcloned into both pJAGint and pJANint (BioGrammatics). Transformation: Electro-competent Pichia GlycoSwitch® SuperMan5 (+) cells were transformed, independently, with the expression vectors after linearization using Pmel; Pmel cuts at a unique site in the AOX1 promoter (pAOX1), targeting integration at the AOX1 locus. Co-transformation was done with both the pJAGaMF-A1AT and pJANaMF-A1AT vectors for each type of modified A1AT ORF. Regardless of ORF sequence inserted, thousands of transformants were selected, and the selected clones were patched and tested on YPD plates with G418 or Nourseothricin (Nat) to determine if they were G418 and/or Nat resistant. Approximately 5% of the clones were resistant to both of these drugs. Individual clones resistant to G418, Nourseothricin, or both were then selected for expression testing. Prior to expression testing of a specific clone, it was also struck on plate culture to allow for the recovery of a single isolated colony.

### Example 2 Expression Testing

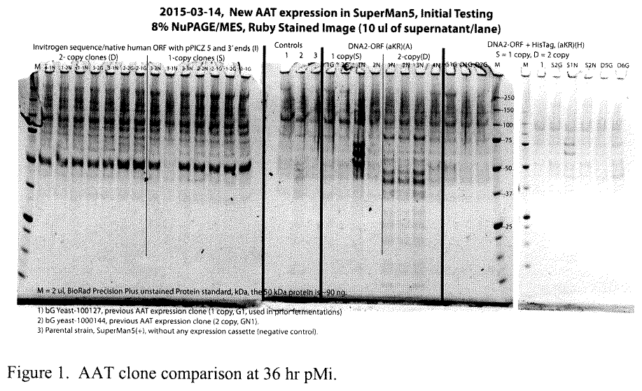

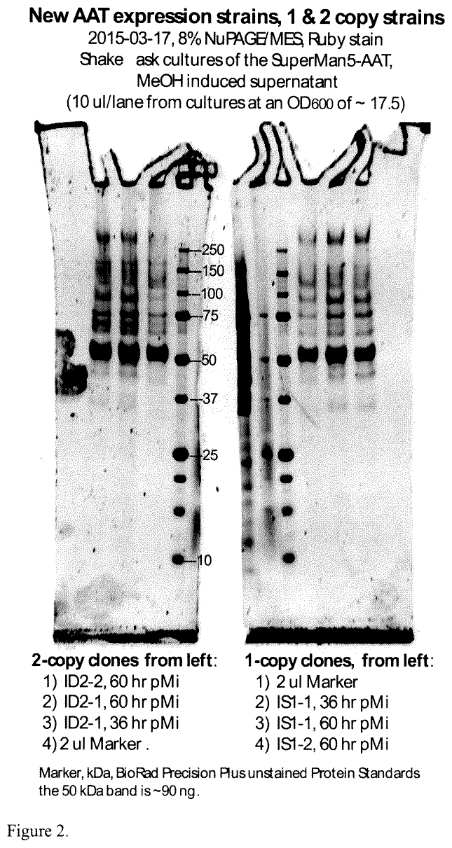

Pichia transformed with select clones were inoculated into Pichia growth media BMGY (1.5% glycerol), grown overnight at 30° C., with 200 rpm shaking, prior to centrifugation, removal of the supernatant and re-suspension of the cells in Pichia induction media BMMY (1% methanol). The methanol concentration was increased to 1% with BMMY (10% methanol) at 24 hr, and every 12 hr thereafter. After 36 hr post methanol induction (pMi), multiple clone cultures from each type of construct were sampled. The supernatant was separated from the cells using centrifugation 30 (˜650× g). The rALAT expression and secretion levels were determined using separation on a 8% polyacrylamide gel electrophoresis (PAGE)/MES (NuPAGE® SDS-PAGE: Thermo Fisher Scientific, NuPAGE® MES SDS Running Buffer is recommended for separating small-to medium-sized proteins and the use of MES buffer allows proteins to run faster than when using MOPS buffer), prior to SYPRO Ruby protein gel staining (Molecular Probes). SYPRO Ruby protein gel staining is a highly sensitive, ready-to-use fluorescent stain for the detection of proteins separated by PAGE) and imaging (FIG. 1). In all cases, except for one, the clones with the hA1AT ORF secreted more rA1AT in the 36 hr post methanol induction (pMi) supernatant (see FIG. 1, left lanes, all but 3-IN have a prominent band at ˜52 kDa). Unlike the DNA 2.0 designed, 2A1AT and 2tAAT, ORFs, or the hA1AT expression clones bG-Yeast-100127 and bG-Yeast-100144 (Controls 1 and 2). Analysis of later time-points indicated that more A1AT protein was secreted expressed and secreted with “2-copy” clones than clones predicted to contain only 1 copy of the expression vector (FIG. 2, left vs. right panel). Furthermore, expression levels at 60 hr pMi appear to be slightly better than at 36 hr. pMi; however, in this case more protein at higher molecular weight is evident (FIG. 2). A1AT levels in these shake flask cultures is estimated to be >25 μg/ml indicating higher cell density fermentation could generate ˜25 mg/liter. Recombinant A1AT (rA1AT) expression clones with the native human A1AT ORF were created and expression levels from shake flask cultures indicate significantly more rA1AT is generated than was made from the bG Yeast-100127 strain which was methanol induced in parallel. Fold higher levels are estimated to be secreted from the strains predicted to have 2 copies of the A1AT ORF. The “2-copy” clone identified as ID2-1G was selected as bG Yeast-100180 and was tested for activity. With positive results, production under fermentation conditions should be performed to generate greater than 10 mg of rA1AT for purification and more detailed testing. Elastase binding in a gel-shift assay demonstrated activity, and purification of ˜20 mg was performed from induced shake-flask supernatant samples after preliminary shake flask induction optimization. In parallel, a 1.5-liter high cell density fermentation was performed to test production in a fermentor; similar gel shift activity was observed with the crude fermentation supernatant with significantly higher levels of rA1AT. Purification of the rA1AT from shake flask induced supernatant was performed by the previously outlined scheme: Anion exchange (Q) clearing, Tangential Flow Filtration/TFF, and subsequent Q, Phenyl Sepharose (PS), and HydroxyAppatite (HA) chromatography. Greater than 25 mg A1AT at >95% purity were generated; the A1AT was active.

### Example 3 Activity Testing

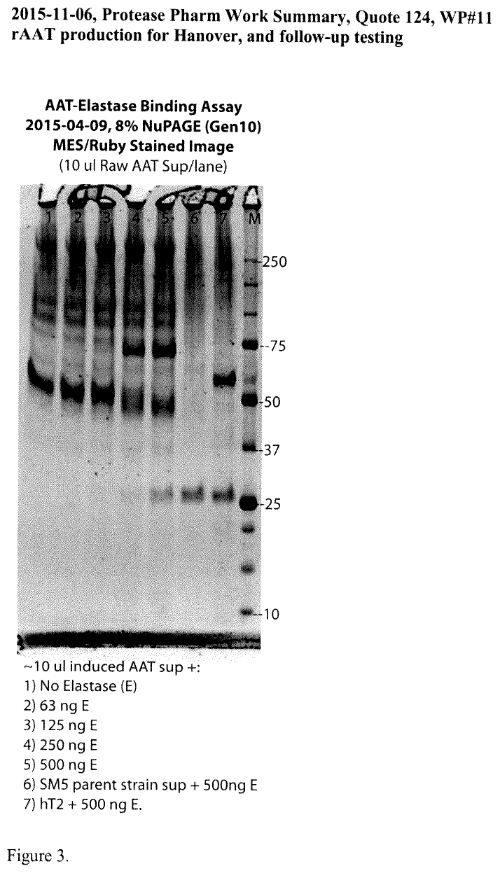

The new, higher producing Pichia rA1AT production strain identified above as ID2-1G as a glycerol stock (30% glycerol in YPD), and designated bG 10 Yeast-100180. bG Yeast-100180 cells were grown overnight in Pichia growth media BMGY (0.75% glycerol), separated from the medium by centrifugation (2000 rpm IEC, 5 min) and re-suspended in Pichia induction media BMMY (1% methanol) were subsequently fed 1/10 of the culture volume with induction media BMMY (containing 10% methanol) at 24 hr and every 12 hr thereafter. Samples were collected at various time points and the supernatant separated from the cells prior to analysis (for example see FIG. 3Y1) with Direct PAGE (NuPAGE/MES Noves) with SYPRO Ruby staining (Molecular Probes). rALAT migrates just above the 50 kDa standard; upon incubation with elastase (Sigma), both the rA1AT and elastase “gel shift”, as predicted by elastase binding the rA1AT and becoming covalently bound to the rA1AT. At the highest concentrations of elastase all of the A1AT is gel shifted (see lanes 4 & 5, FIG. 3). PAGE analyses after Endo H treatment (1 hr at 37° C., NEB protocol) Western analyses following PAGE, transfer to PDVF membranes (BioRad), TBTS incubations with primary antibody (Abcam: anti A1AT, Ab #179443, a Rabbit monoclonal Ab, 1/2500) and secondary antibody (Goat-anti Rabbit, Alk Phos conjugate 2ndary, 1/5000 Abcam).

### Example 4 Shake Flask Induction Optimization and Production

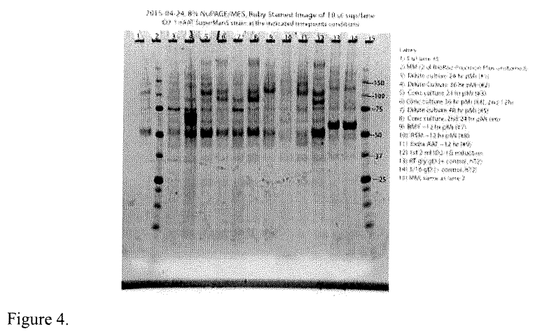

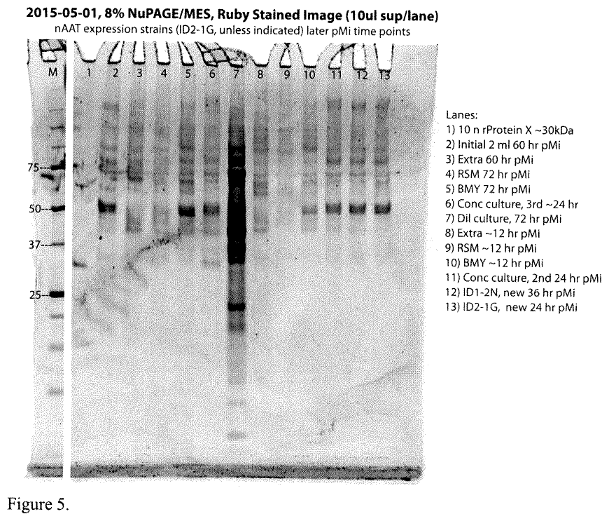

Small shake flask cultures containing ID2-1 nA1AT SuperMan5 strain were tested with different mediums and Pichia induction media BMMY (1% methanol) vs. RSM (a proprietary medium from Mediomics); at different induction time-points (sampled 12-48 hr post Methanol induction, pMi); at different cell densities at the time of shift from glycerol to methanol (2-20 OD600). Supernatant samples were compared by SDS-PAGE-Ruby-stained analyses (as shown in FIG. 4 and FIG. 5). Pichia growth media BMGY (1.5% glycerol) and Pichia induction media BMMY 5 (1% methanol) with the cell culture at high cell density at the time of shift to methanol induction, and methanol induction for 36 hr was selected as the shake flask induction protocol. Subsequently, larger shake flask cultures were induced using this protocol and pooled for purification.

### Example 5 Purification Using Serial Chromatographic Techniques

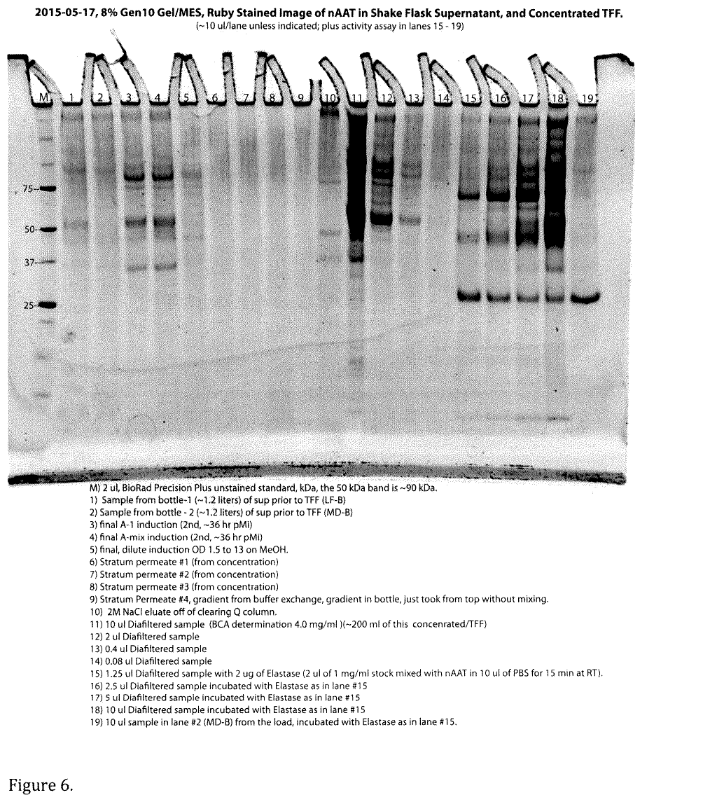

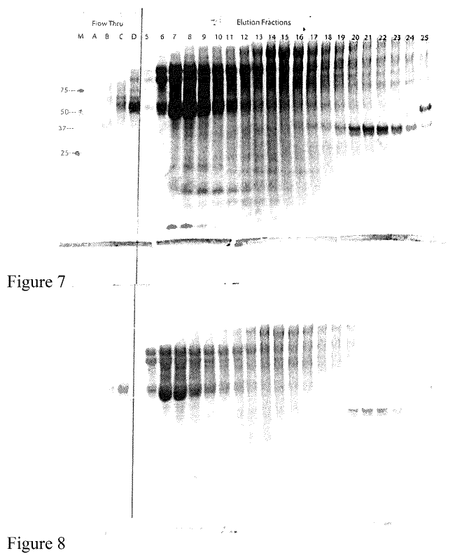

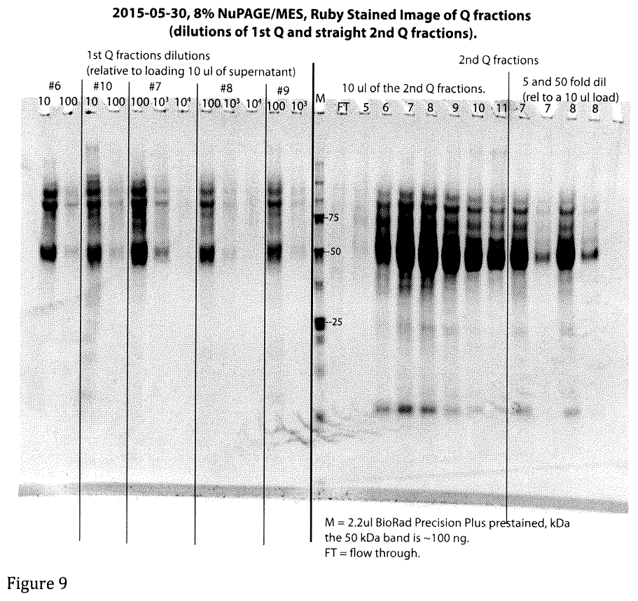

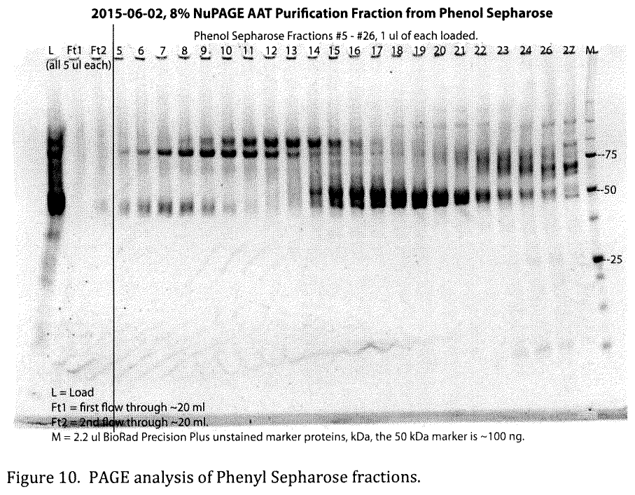

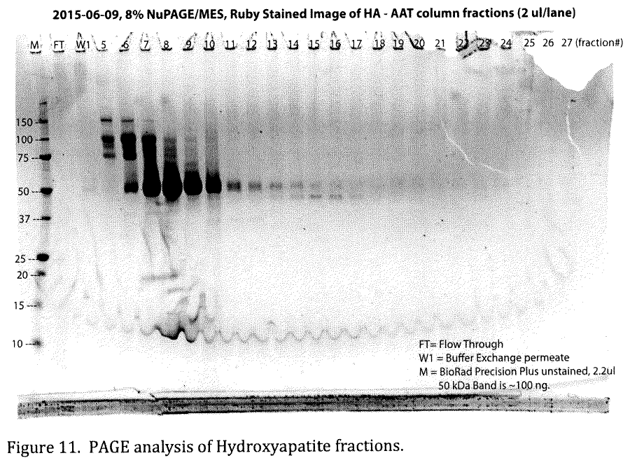

Approximately 2 liters of pooled methanol induced supernatant were subject to centrifugation to remove any residual Pichia cells. Then the clarified A1AT containing supernatant was passed through a 100 ml column with Q Sepharose matrix (BioRad, Macro-Prep High Q, Strong Anion exchange support) which had been washed with approximately 200 ml of 150 mM NaCl, 10 mM Tris (pH 8), 5 mM EDTA. The presence of a high concentration of phosphate and other salts in the supernatant prevents the A1AT from binding this matrix, however, significant contaminating material from the supernatant is removed as was evidenced by the brown color change of the Q Sepharose. This preclearing step using Q Sepharose facilitated the subsequent steps of buffer exchange and Tangential Flow Filtration (Kros Flo, Research II/TFF system, Spectrum Labs, with 10 kDa Spectrum filter TFF) and a buffer exchange with 10 mM Tris (pH 8), 5 mM EDTA, 1 mM beta-mercaptoethanol. Glycerol was added to a final 15% and sample was frozen at −80° C. TFF also concentrated the sample to approximately 200 ml (10× concentration). The total protein concentration of the sample, post TFF, was estimated to be ˜4 mg/ml by BCA protein determination (200 ml total, as shown in FIG. 6, lanes 11-14 for 10-0.08 μl of sample). Additionally, the sample, post TFF was determined to still have Elastase binding activity (FIG. 6, lanes 15-19). Anion Exchange Chromatograpy (HiTrap Capto Q): Following Tangential Flow Filtration (TFF), 100 ml of sample was loaded onto 2 HiTrap Q columns (HiTrap Capto Q, GE HealthCare Biosciences, Pittsburgh, PA, USA), anion exchange columns, in series (washed with 10 mM Tris (pH 8), 5 mM EDTA). The column was washed with 5 column volumes of 10 mM Tris (pH 8) 5 mM EDTA, prior to a gradient elution over 5 column volumes (50 ml) from 0 to 500 mM NaCl, with a final elution step to 1 M NaCl and 2 ml fractions were collected (as shown FIG. 7). The remaining 100 ml of the TFF sample was subsequently purified on the same HiTrap Q column, as with the initial 100 mls (FIG. 8). There was some of the rA1AT in the last part of the “flow through” indicating a larger column might have captured more rA1AT. A closer look at the peak fractions indicated a similar sized rALAT on both sides of the peak fractions (see FIG. 8, fractions 6 and 10 next to each other, 1st Q fractions). FIG. 8, fractions 7-9 from both HiTrap Q chromatography steps were pooled for the next chromatography step (phenyl sepharose separation). Phenyl Sepharose Chromatography: The pooled fractions from the HiTrap Q anion exchange chromatography step were brought to 2 M ammonium sulfate by adding an equal volume of 4 M ammonium sulfate, 5 mM sodium phosphate pH 7.0. FIG. 9 shows concentrated and diluted HiTrap Q column fractions from the second application. The samples were run over a 5 ml HiTrap Phenyl HP column (GE HealthCare BioSciences, Pittsburgh, PA, USA) and the rA1AT was eluted with a gradient of ammonium sulfate from 2-0 M over 12 column volumes (60 ml). Fractions samples were analyzed by SDS-PAGE and SYPRO Ruby staining (shown in FIG. 10). Fractions 15-21 were pooled for use in the next chromatographic separation step. Hydroxyapatite Chromatography: The pooled fractions 15-21 were combined and using a Amicon Ultra-15 Centrifugal Filters (Ultracel-10 kDa cut off) buffer exchanged to 5 mM sodium phosphate (2.5 mM monobasic, 2.5 mM dibasic), 200 μM CaCl2. Using a Bio-Scale™ CHT™ Type 1 column chromatography (5 ml, BioRad, CHT ceramic hydroxyapatite, Type I 10 μm support, which has high affinity for basic proteins and lower affinity for acidic proteins). Samples were loaded and washed with 5 column volumes of 5 mM sodium phosphate (2.5 mM monobasic, 2.5 mM dibasic), eluted with 500 mM sodium phosphate (250 mM monobasic, 250 mM dibasic) using 10 column volumes (50 ml). Both buffers also contained 200 UM CaCl2. Fractions were analyzed by SDS-PAGE and SYPRO Ruby staining (as shown in FIG. 11). Using this method, greater than 25 mg rA1AT with a purity of >95% and was shown to bind elastase. This purified rAAT was determined to be stable for more than 2 weeks at 37° C.

### Example 6 Analyses of the Purified RA1AT

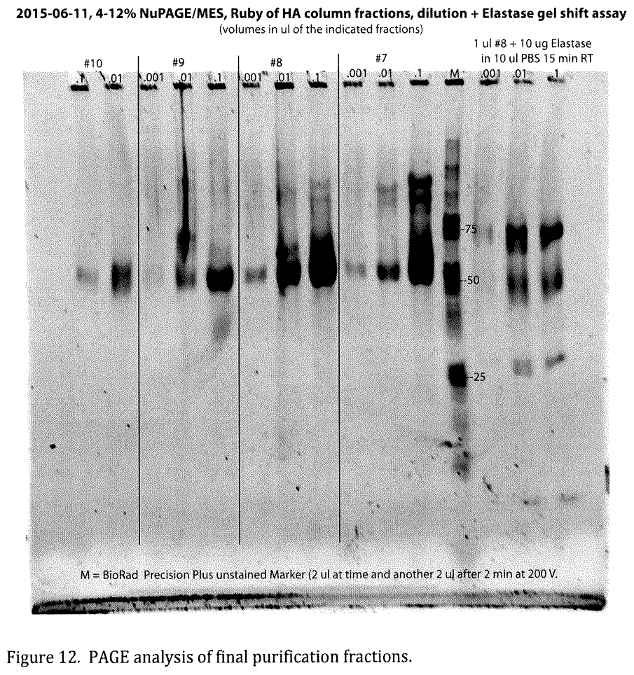

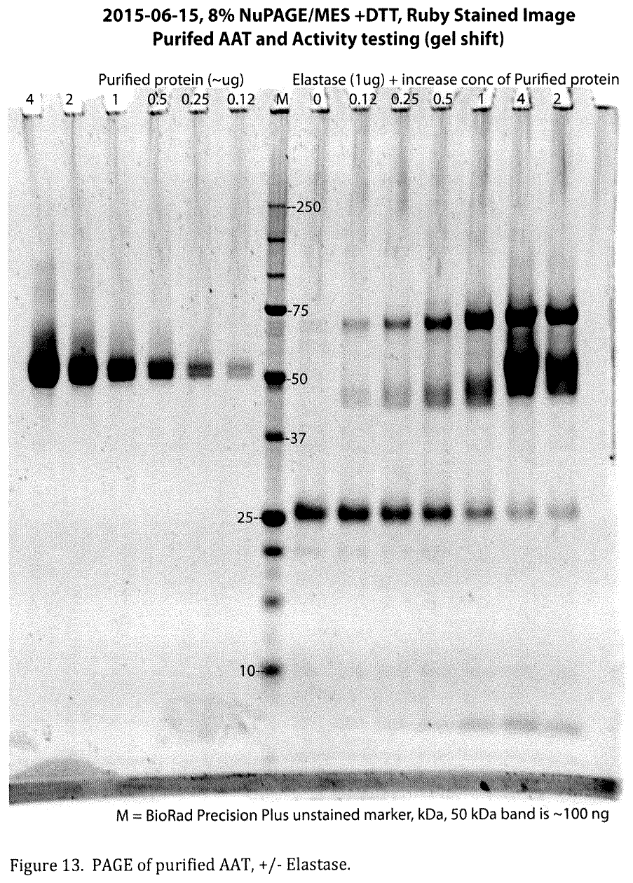

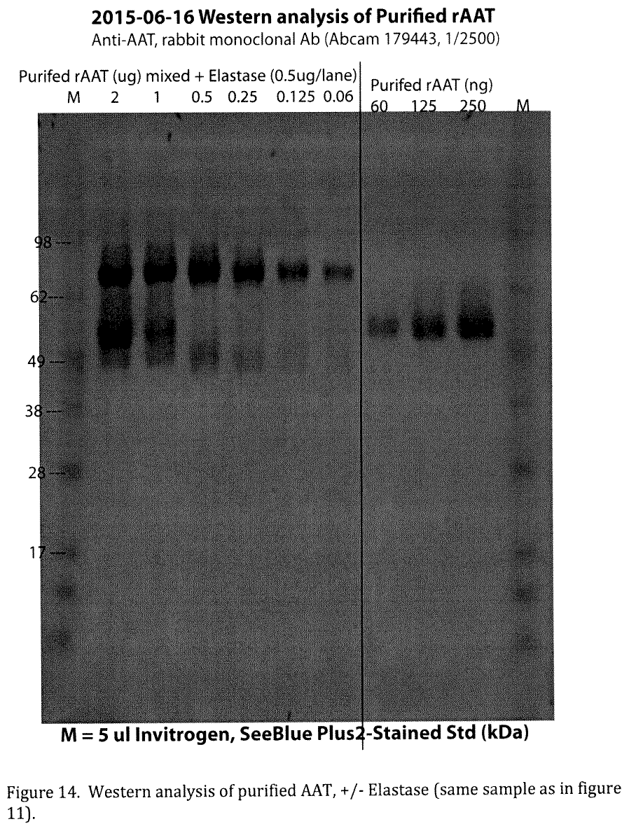

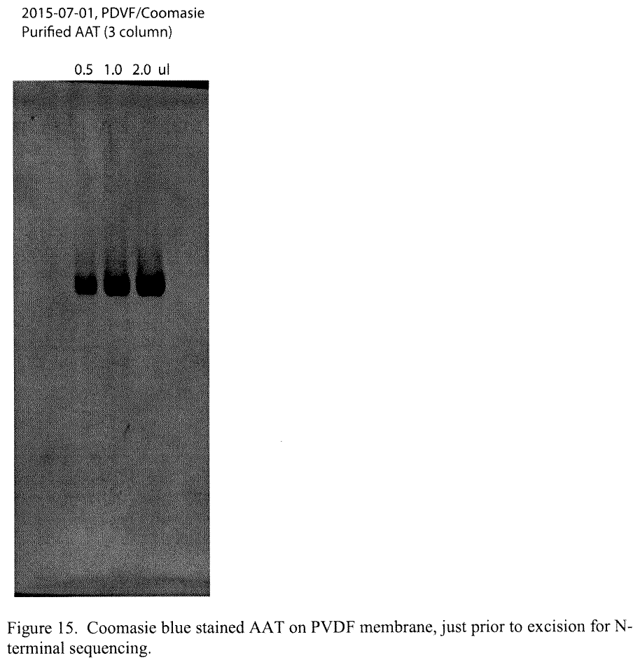

As shown in FIG. 12, fractions #7-10 eluted from the hydroxyapatite column (Bio-Scale™ CHT™ Type 1 column) were analyzed separately and the size of the rA1AT as determined by migration in the 8% NuPAGE gel analysis indicated that all of the rA1AT in the peak fractions were of similar size (˜55 kDa Fractions #8 and 9 were chosen to be pooled for use as a final purified rA1AT preparation for analysis. It should be noted that although fraction 7 contained approximately 30% of the rA1AT, it was not included in the final rALAT preparation for analysis as it contained a higher molecular weight contaminant which has yet to be characterized. A1AT is known to bind elastase and an elastase gel shift assay can be used to identify the presence of A1AT activity. For example, volumes of fraction #8 were added to 10 μg of elastase in 10 μl of PBS and incubated for 15 min at room temperature. This assay demonstrated that the rA1AT in fraction #8 had the expected elastase binding activity (as shown in FIG. 12 in the far-right lanes). On further examination using PAGE analysis, this final purified rALAT preparation appeared to be 95% pure (see the concentrations of the purified protein in the first 6 lanes of FIG. 13). BCA and Bradford protein determinations on this final purified rA1AT preparation indicated that the concentration is approximately ˜5 μg/μl. However, in comparing the purified rA1AT to the marker protein standards (FIG. 13, Lane M) not this concentrated (compare the 50 kDa band in the BioRad marker which contains approximately 100 ng, to the diluted rA1AT (FIG. 13). Additionally, two forms of the rALAT became apparent and this is best seen in the lower dilutions of the rALAT directly run in FIG. 11, lanes containing 0.5-0.12 μg. Both forms also appear to be recognized by the A1AT antibodies (FIG. 14). This suggests that these differences in the two forms can be the result of degradation at one end of the rA1AT or alternatively to some post-translational modification like glycosylation. Elastase gel shift assay results obtained using 1 μg of elastase combined with increasing concentrations (0, 0.12, 0.25, 0.5, 1, 4, and 2 μg) of final purified rALAT preparation are shown to the right of the Marker Lane (M) in FIG. 13 demonstrated that this purified rALAT preparation was active. Western blot analysis was done on final purified rALAT preparation samples that were separated by PAGE as previously described, and blotted to PVDF membrane is shown in FIG. 14. In this immunoblot a 1/2500 dilution of rabbit monoclonal anti-A1AT antibody (Abcam 179443) was used to confirm the identify A1AT protein in the final purified rALAT preparation (shown in the right lanes are concentrations of 60 ng, 125 ng, and 250 ng and shown on the left lanes of FIG. 14, is the expected shift associated with elastase binding when decreasing concentrations (2 μg. 1 μg, 0.5 μg, 0.25 μg, 0.125 μg, 0.06 μg) of rALAT was added to 0.5 μg of Elastase. N-Terminal Protein Sequencing: In addition, purified rALAT preparation samples separated by PAGE as previously described, and transferred to PVDF membrane were imaged after coomassie blue staining (FIG. 15) and were excised from the membrane for Nterminal sequencing. The rALAT bands was cut in half, in order to separate the lower and faster migrating bands for separate analysis. The N-terminal sequencing was performed at Tufts University Core Facility in the Physiology Department (Boston, MA, USA). It was determined that the first 8 N-terminal amino acids of the top (slower 20 migrating/larger) form of rA1AT were E, D, P (G), Q (V), G(S), D (L), A, A and for the bottom (faster migrating/smaller) form of rA1AT they were E, D, P, Q, G, D, A, A. These are both consistent with the first 8 N-terminal amino acids of the known native human A1AT protein: E, D, P, Q, G, D, A, A and in both cases indicates that the N-terminus of the rA1AT present in both bands is intact and not degraded and supports the conclusion that these 2 25 bands represent different glycoforms. A glycoform is an isoform of a protein that differs only with respect to the number or type of attached glycan.

### Example 7 Stability Testing

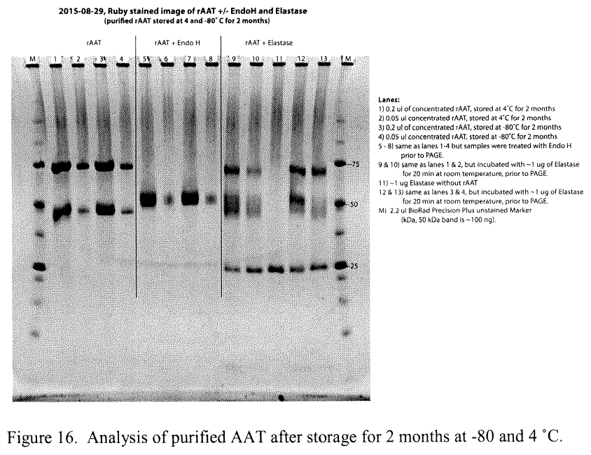

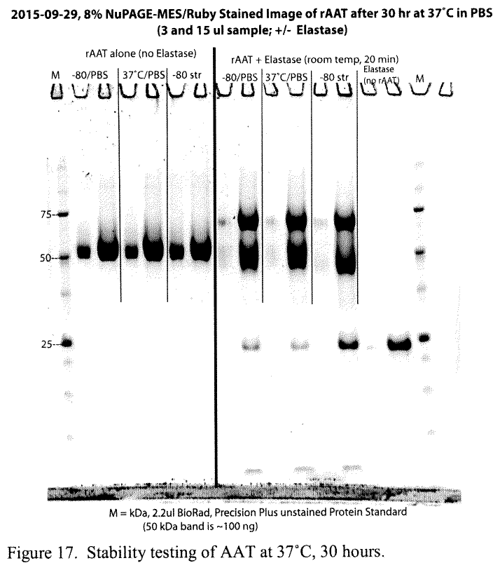

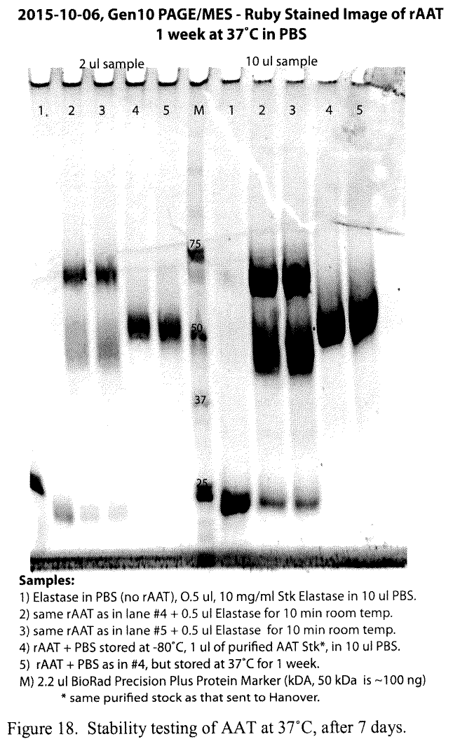

Preliminary stability testing of rALAT samples (BioGrammatics) 30 are shown in FIG. 16. Aliquots of the purified rALAT samples were stored at −80° C., aswell as 4° C. for 2 months. After which these rA1AT appeared similar, both in size before and after EndoH treatment (lanes 1-8, FIG. 16); and Elastase binding activity levels (FIG. 16, lanes 9-13) In addition, to accelerate sample deterioration, some purified rA1AT samples were placed at 37° C. after dilution into 1× phosphate buffered saline (PBS). It was determined that samples subject to either 30 hr at 37° C. (results shown in FIG. 17) and those samples subject to 7 days at 37° C. (results shown in FIG. 18) showed no significant change is size (as determined by PAGE) or elastase binding activity.

### Example 8 Larger Scale Fermentation

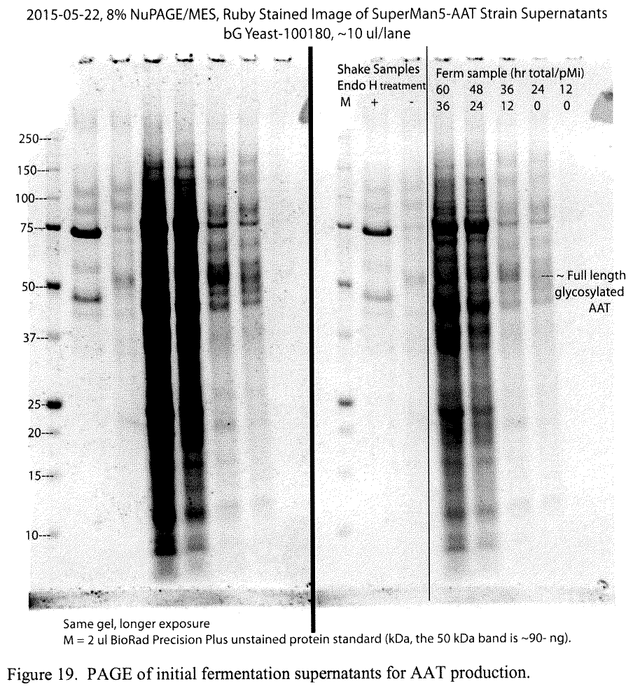

FIG. 19: PAGE of initial fermentation supernatants for A1AT production.

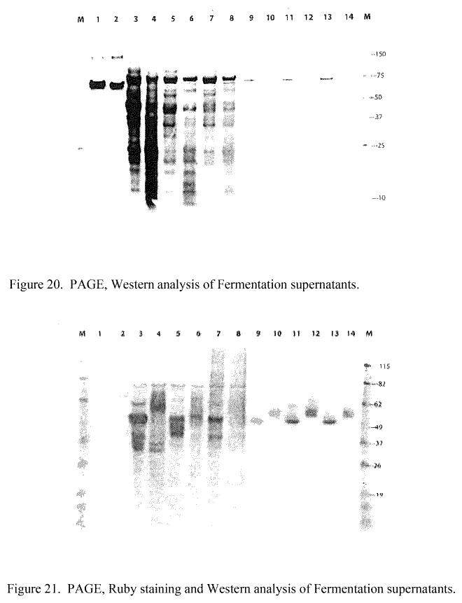

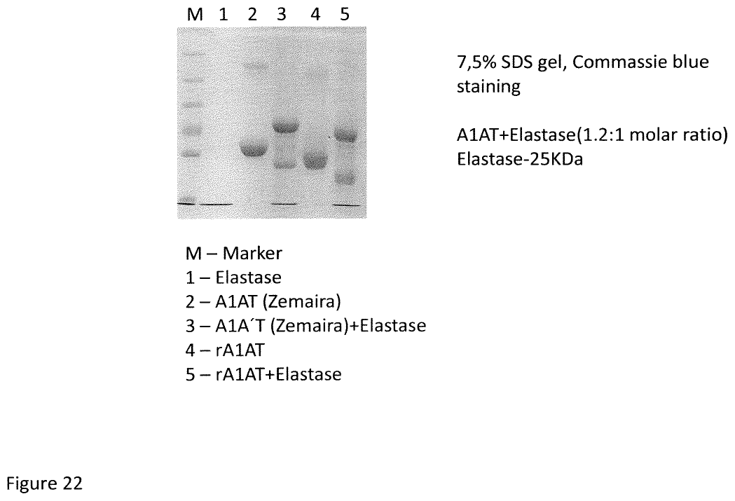

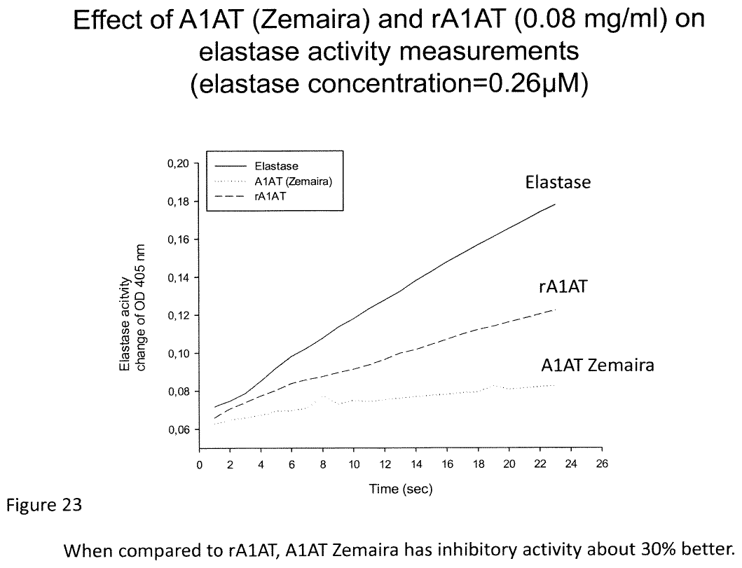

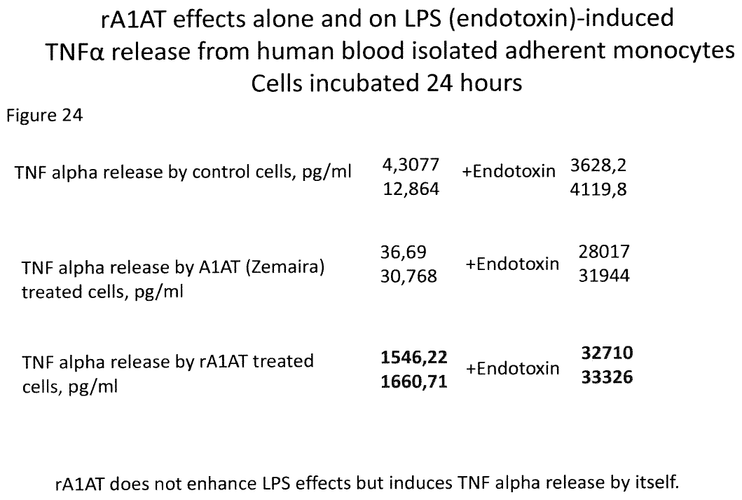

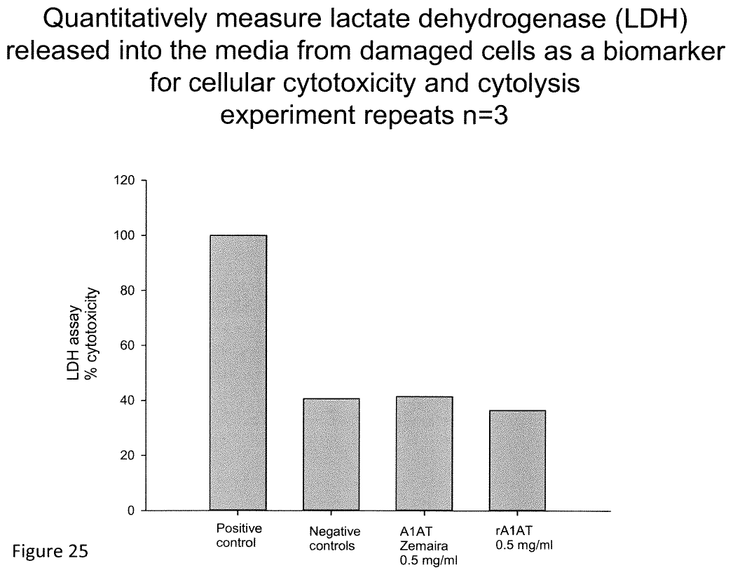

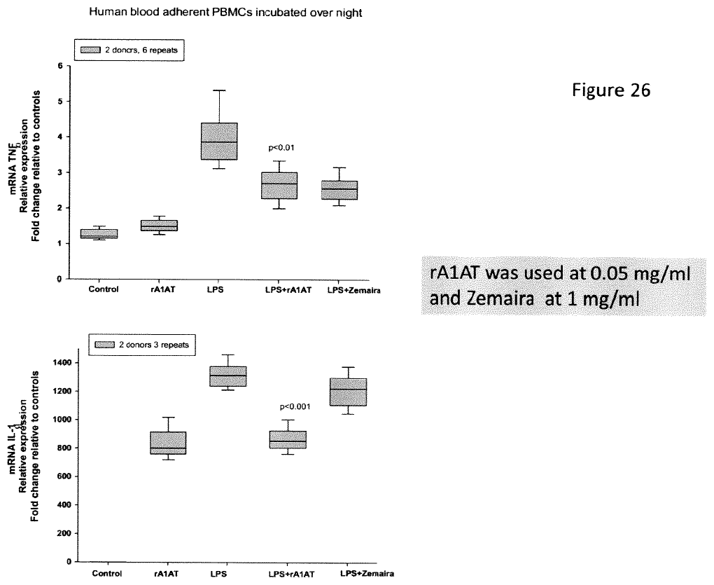

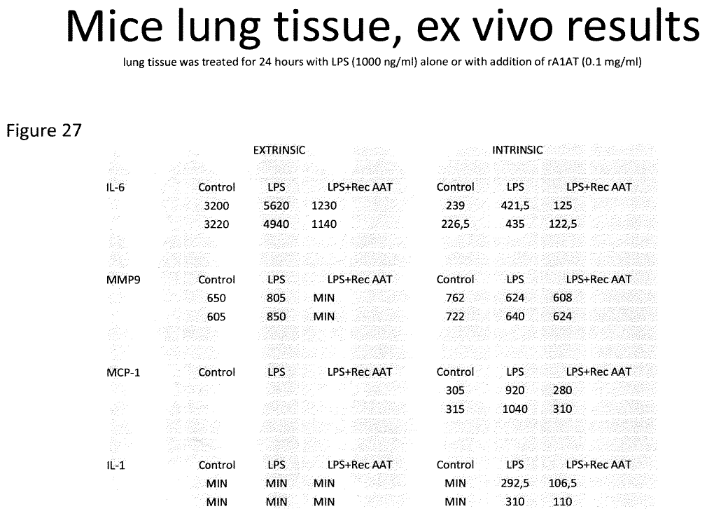

FIG. 20: PAGE and Western analysis of Fermentation supernatants.

FIG. 21: PAGE and Ruby staining of Fermentation supernatants.

Sample Volumes/mass:
1) 0.6 ng control Human AAT from ELISA kit+Endo H*
2) same as lane #1, but without Endo H
3) 36 hr pMi, Cornell Ferm sample (fresh thawed sup), +Endo H. (R=11 μl, W=7 μl)
4) Same as lane #3, but without Endo H.
5) Same as in lane #3, but sample was stored at 4° C. ˜3 weeks instead of −80° C. +Endo H
6) Same as in lane #5, without Endo H
7) 24 hr pMi, Ferm samples, stored at 4° C. 3 weeks, +Endo H. (R=11 μl, W=7 μl)
8) same as lane #7, but without Endo H
9) Purified AAT, post HA column+glycerol, Fraction #10, +Endo H (loaded 0.5 μl to Ruby 0.2 μl to Western)
10) same as lane #9 but without Endo H
11) Purified AAT, post HA column+glycerol, Fraction #7, +Endo H (loaded 0.2 μl to Ruby. 0.08 μl to Western)
12) #11, without Endo H
13) Purified AAT, post HA column+glycerol, Fraction #8&9 pooled, best samples, +Endo H (loaded 0.1 μl to Ruby 0.04 μl to Western)
14) Same as lane #13 but without Endo H.
M-FIG. 20) 2.2 μl BioRad Precision Plus unstained marker (kDa, 50 kDa brand is ˜100 ng)
M-FIG. 21) 6 μl BenchMark, Invitrogen, prestained marker (kDa).
Endo H treatment for 1 hr at 37° C. as recommended by the manufacturer (NEB).

### Example 9 Glycan Profiling

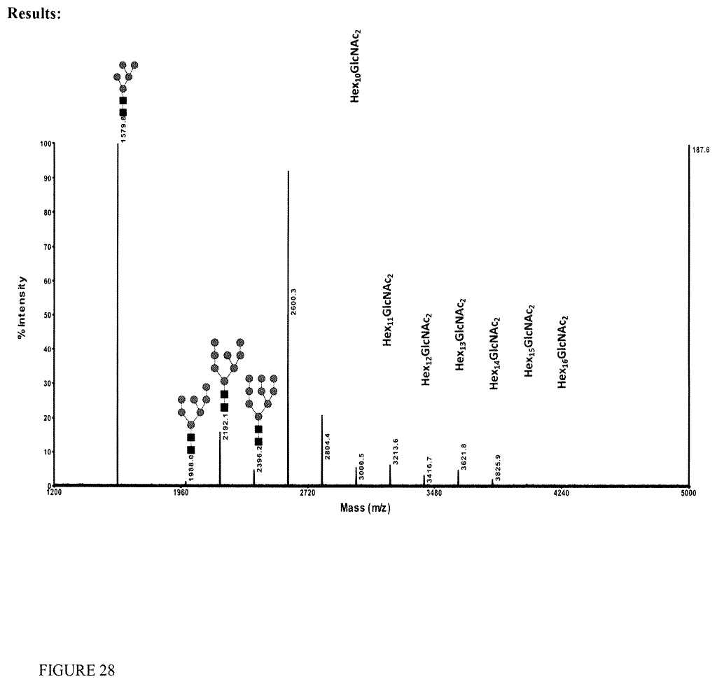

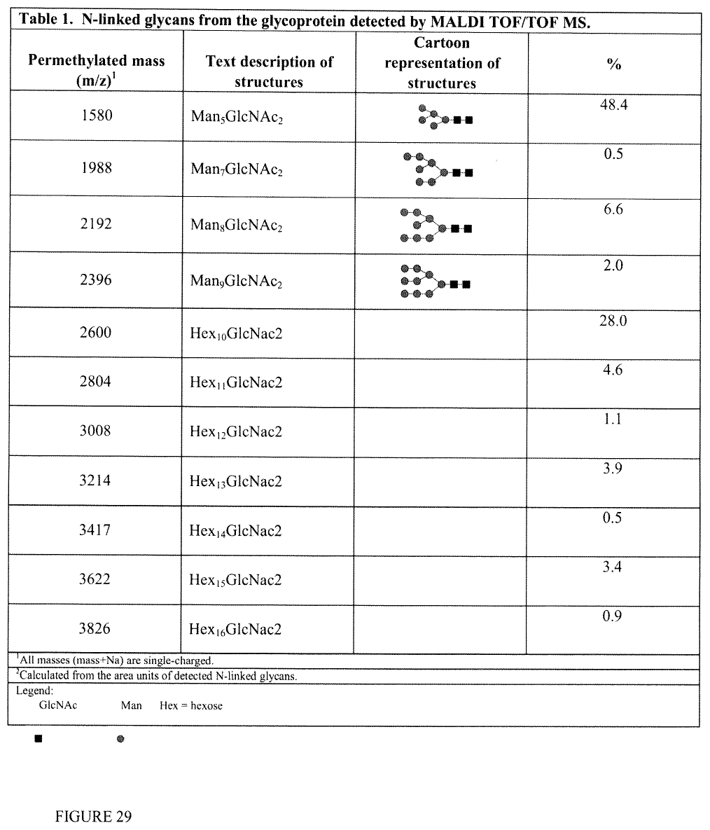

N-linked glycans profiling by MALDI-TOF/TOF MS was done at the Complex Carbohydrate Research Center (University of Georgia) the results are shown in FIGS. 28 and 29. The sample was dialyzed against deionized and nanopure water over a 24-hr period using a membrane with 4000 MWCO. The water was completely replaced 6 times with the last two being nanopure water. To release the N-linked glycans, a weighed amount of the dialyzed sample (˜600 μg) was digested with trypsin in Tris-HCl buffer overnight. After protease digestion, the sample was passed through a C18 sep pak cartridge, washed with a low concentration of acetic acid and the glycopeptides were eluted with a blend of isopropanol in low concentration acetic acid. The eluate was dried and treated with PNGase F to release the N-linked glycans. Per-O-Methylation Of N-Linked Glycans: The N-linked glycans were permethylated for structural characterization by mass spectrometry (Anumula and Taylor, 1992). Briefly, the dried eluate was dissolved with dimethyl sulfoxide and methylated with NaOH and methyl iodide. The reaction was quenched with water and per-O-methylated carbohydrates were extracted with methylene chloride and dried under N₂. Profiling By Matrix-Assisted Laser-Desorption Time-Of-Flight Mass Spectrometry (Maldi-Tof/Tof Ms): The permethylated glycans were dissolved with methanol and crystallized with 2,5-dihyroxybenzoic acid (DHBA) matrix. Analysis of glycans present in the samples was performed by MALDI-TOF/TOFMS using AB SCIEX TOF/TOF 5800 (Applied Biosystem MDS Analytical Technologies).

### Example 10 Bioactivity

Bioactivity of recombinant rA1AT glycoprotein: Testing of rA1AT in vitro, in human cell culture models and in mice lung precise cut slides, ex vivo. For these tests rA1AT was re-suspended in 250 μl of sterile double distilled water. Elastase activity assay: Elastase activity studies were carried out in 0.1M Tris's buffer, 15 pH, 8.0. rALAT was diluted to a final concentration of 0.08 mg/ml. Briefly, rA1AT was incubated with elastase in 1:2.6 molar ratio at 37° C. for 5 min. Afterwards, was added Nsuccinyl-Ala-Ala-Ala-p-nitroanilide and elastase activity was measured by spectrophometry at 405 nm for 3 min. Blank contained substrate alone.

### Examples 11 to 20 First Scale Up

The above Examples 1 through 10 are repeated with scale up changes as follows: Shake reactors, centrifuges and other pilot plant equipment replace flasks and other labware, and the quantities of constituents are scaled by a factor of 50. Further, there is a continuous feed input to the reactor, including the ID2-1 nA1AT SuperMan5 strain described above into the reactor containing the described Pichia induction media in methanol. Start-up constituents are fed proportionately. The reactor includes feed lines and output lines, and output is released in a steady flow, for subsequent product purification and isolation. The yeast is self-perpetuating in that the process in extracellular expression. The recovery process includes maintaining the vitality of the Pichia Yeast (meaning, the yeast is not killed in the isolation and purification of the desired product). Results compare to the lab processing of Examples I through 10 above.

### Examples 21 to 30 Second Scale Up

The above Examples 1 through 10 are repeated with scale up changes as follows: Shake reactors, centrifuges and other pilot plant equipment replace flasks and other labware, and the quantities of constituents are scaled by a factor of 50. Further, there is a continuous feed input to the reactor, including the ID2-1 nA1AT SuperMan5 strain described above into the reactor containing the described Pichia induction media in methanol. Start-up constituents are fed proportionately. The reactor includes feed lines and output lines, and output is released in a steady flow, for subsequent product purification and isolation. The yeast is self-perpetuating in that the process in extracellular expression. The recovery process includes maintaining the vitality of the Pichia Yeast (meaning, the yeast is not killed in the isolation and purification of the desired product). In these examples, the yeast that is removed from which product is recovered, is recycled into the reactor. Results compare to the lab processing of Examples 1 through 10 above.

### Examples 31 to 40 Third Scale Up

The above Examples 1 through 10 are repeated with scale up changes as follows: Shake reactors, centrifuges and other pilot plant equipment replace flasks and other labware, and the quantities of constituents are scaled by a factor of 50. Further, there is a continuous feed input to the reactor, including the ID2-1 nA1AT SuperMan5 strain described above into the reactor containing the described Pichia induction media in methanol. Start-up constituents are called proportionately. The reactor includes feed lines and output lines, and output is released in a steady flow, for subsequent product purification and isolation. The yeast is self-perpetuating in that the process in extracellular expression. The recovery process includes maintaining the vitality of the Pichia Yeast (meaning, the yeast is not killed in the isolation and purification of the desired product). In these examples, the yeast that is removed from which product is recovered, is recycled into the reactor, and additional yeast/medium feed is inputted into the reactor. Flow rates of constituents are adjusted to create a steady state continuous flow process and Results compare to the lab processing of Examples 1 through 10 above.

What is claimed is:

1. A method of manufacturing recombinant glycoprotein comprising human alpha-1 antitrypsin having non-native glycosylation with a substantially homogeneous population of N-glycans, in an extracellular expression process, which comprises:
(a) expressing a polynucleotide sequence encoding human alpha-1 antitrypsin polypeptide in an engineered AAT expression strain comprising one of a mutant OCH1 gene and a mannosidase gene, wherein the engineered AAT expression strain does not contain a WT OCH1 gene wherein said AAT expression strain is an expression vector that is a Pichia Pastoris Yeast strain,
(b) isolating supernatant; and
(c) purifying the non-natively glycosylated AAT polypeptide from the supernatant to provide the recombinant glycoprotein and less than 20% Man7GlcNAc2, Man8GlcNAc2 and Man9GlcNAc2,
wherein said method of manufacturing, including said isolating and said purifying, is performed while maintaining the vitality of said Pichia Pastoris Yeast.

2. The method according to claim 1, wherein said glycoprotein is SEQ ID NO: 18.

3. The method according to claim 1, wherein said non-native glycosylation comprises a population of at least 80% homogeneous N-glycans.

4. The method according to claim 1, wherein Man7GlcNAc2, Man8GlcNAc2, and Man9GlcNAc2 N-glycans comprise less than about 10% of the total N-glycans.

5. The method according to claim 1, wherein Man7GlcNAc2, Man8GlcNAc2, and Man9GlcNAc2 N-glycans comprise less than about 5% of the total N-glycans.

6. The method according to claim 1, wherein Man7GlcNAc2, Man8GlcNAc2, and Man9GlcNAc2 N-glycans comprise less than about 1% of the total N-glycans.

7. A method of manufacturing recombinant glycoprotein comprising human alpha-1 antitrypsin having non-native glycosylation with a substantially homogeneous population of N-glycans, in an extracellular expression process, which comprises:
(a) in a continuous flow process, expressing a polynucleotide sequence encoding human alpha-1 antitrypsin polypeptide in an engineered AAT expression strain comprising one of a mutant OCH1 gene and a mannosidase gene, wherein the engineered AAT expression strain does not contain a WT OCH1 gene wherein said AAT expression strain is an expression vector that is a Pichia Pastoris Yeast strain;
(b) isolating supernatant; and
(c) purifying the non-natively glycosylated AAT polypeptide from the supernatant to provide the recombinant glycoprotein product with less than 20% Man7GlcNAc2, Man8GlcNAc2 and Man9GlcNAc2,
wherein said method of manufacturing, including said isolating and said purifying, is performed while maintaining the vitality of said Pichia Pastoris Yeast.

8. The method according to claim 7, wherein said glycoprotein is SEQ ID NO: 18.

9. The method according to claim 7, wherein said non-native glycosylation comprises a population of at least 80% homogeneous N-glycans.

10. The method according to claim 7, wherein Man7GlcNAc2, Man8GlcNAc2, and Man9GlcNAc2 N-glycans comprise less than about 10% of the total N-glycans.

11. The method according to claim 7, wherein Man7GlcNAc2, Man8GlcNAc2, and Man9GlcNAc2 N-glycans comprise less than about 5% of the total N-glycans.

12. The method according to claim 7, wherein Man7GlcNAc2, Man8GlcNAc2, and Man9GlcNAc2 N-glycans comprise less than about 1% of the total N-glycans.

13. The method according to claim 7, wherein said method of manufacturing is performed in at least one reactor wherein said reactor includes feed lines and output lines, and output is released in a steady flow, for subsequent product purification and isolation.

14. The method according to claim 7, wherein said method of manufacturing is performed in at least one reactor wherein said reactor includes feed lines and output lines, and output is released in a steady flow, for subsequent product purification and isolation and feed lines include continuous feed of at least said engineered AAT expression strain.

15. The method according to claim 7, wherein said method of manufacturing is performed in at least one reactor wherein said reactor includes feed lines and output lines, and output is released in a steady flow, for subsequent product purification and isolation and feed lines include continuous feed of at least said engineered AAT expression strain and a separate continuous feed of said Pichia Pastoris Yeast.

16. The method according to claim 7, wherein said method of manufacturing is performed in at least one reactor wherein said reactor includes feed lines and output lines, and output is released in a steady flow, for subsequent product purification and isolation and feed lines include continuous feed of at least said engineered AAT expression strain and output lines include purification and isolation of the recombinant glycoprotein product, wherein output Pichia Pastoris Yeast is at least in part recycled back to said reactor.

17. The method according to claim 7, wherein said method of manufacturing is performed in at least one reactor wherein said reactor includes feed lines and output lines, and output is released in a steady flow, for subsequent product purification and isolation and feed lines include continuous feed of at least said engineered AAT expression strain and a separate continuous feed of said Pichia Pastoris Yeast, and output lines include purification and isolation of the recombinant glycoprotein product, wherein output Pichia Pastoris Yeast is at least in part recycled back to said reactor.


SEQUENCE LISTING (22 sequences)

SEQ ID NO: 1 - protein, 418 aa, Homo sapiens
  REGION 1..418: note = Description of sequence: human alpha 1 antitrypsin precursor
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 396 aa, Homo sapiens
  REGION 1..396: note = Description of sequence: human alpha 1 antitrypsin, mature
HVEDPQGDAAQKTDTSHHDQEHSTFNKITPSLAEFAFSLYRQLAHQSNSTNIFFSPVSIATAFAMLSLGTKADTHSEILEGLNFNLTEIPEAQIHEGFQELLHTLNKPDSQLQLTTGNGLFLNKSVKVVDKFLEDVKKLYHSEAFSVNFEDTEEAKKQINNYVEKGTQGKIVDLVKELDRDTVFALVNYIFFKGKWERPFEVEATKEEDFHVDQATTVKVPMMRRLGMFNIYHCEKLSSWVLLMKYLGNATAIFFLPDEGKLQHLENELTHDIITKFLENENRRSANLHLPKLAITGTYDLKTVLGHLGITKVFSNGADLSGVTEDAPLKLSKAVHKAVLTIDEKGTEAAGAMFLEAIPMSIPPEVKFNKPFVFLMIEQNTKSPLFMGKVVNPTQK

SEQ ID NO: 3 - protein, 398 aa, Homo sapiens
  REGION 1..398: note = Description of sequence: human alpha 1 antitrypsin precursor
VSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 4 - protein, 388 aa, synthetic construct
  REGION 1..388: note = Description of sequence: mutant OCH1 alpha-1,6-mannosyltransferase
MRSDLTSIIVFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 5 - protein, 667 aa, synthetic construct
  REGION 1..667: note = Description of sequence: alpha-1,6-mannosidase
MYRISPIGRKSNFHSREKCLIGLVLVTLCFLCFGGIFLLPDNFGSDRVLRVYKHFRKAGPEIFIPAPPLAAHAPHRSEDPHFIGDRQRLEQKIRAELGDMLDEPPAAGGGEPGQFQVLAQQAQAPAPVAALADQPLDQDEGHAAIPVLAAPVQGDNAASQASSHPQSSAQQHNQQQPQLPLGGGGNDQAPDTLDATLEERRQKVKEMMEHAWHNYKLYAWGKNELRPLSQRPHSASIFGSYDLGATIVDGLDTLYIMGLEKEYREGRDWIERKFSLDNISAELSVFETNIRFVGGMLTLYAFTGDPLYKEKAQHVADKLLPAFQTPTGIPYALVNTKTGVAKNYGWASGGSSILSEFGTLHLEFAYLSDITGNPLYRERVQTIRQVLKEIEKPKGLYPNFLNPKTGKWGQLHMSLGALGDSYYEYLLKAWLQSGQTDEEAREMFDEAMLAILDKMVRTSPGGLTYVSDLKFDRLEHKMDHLACFSGGLFALGAATRQNDYTDKYMEVGKGITNTCHESYIRAPTQLGPEAFRFSEAVEARALRSQEKYYILRPETFESYFVLWRLTHDQKYRDWGWEAVLALEKHCRTAHGYCGLRNVYQQEPQKDDVQQSFFLAETLKYLYLLFSDDSVLPLDEWVFNTEAHPLPIKGANAYYRQAPVTLPVSNAS

SEQ ID NO: 6 - DNA, 1600 bp, synthetic construct
  misc_feature 1..1600: note = Description of sequence: alpha 1 antitrypsin ORF sequence
  source 1..1600: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaagagagaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataagcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaa

SEQ ID NO: 7 - DNA, 4717 bp, synthetic construct
  misc_feature 1..4717: note = Description of sequence: predicted pPICZalphaA-A1AT vector sequence
  source 1..4717: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaagagagaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataagcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 8 - DNA, 750 bp, synthetic construct
  misc_feature 1..750: note = Description of sequence: Gblock with overlap at pAOX1
  source 1..750: mol_type = other DNA
gagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaagagagaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaa

SEQ ID NO: 9 - DNA, 955 bp, synthetic construct
  misc_feature 1..955: note = Description of sequence: Gblock with overlap at transcription termination side
  source 1..955: mol_type = other DNA
gaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataagcggccgccagctttctagaacaaaaactcatctcagaagaggatctgaatagcgccgtcgaccatcatcaccatcatcattgagtttgtagccttagacatgactgttcctcagttcaagttgggcacttacgagaagaccggtcttgctagattctaatcaagaggatgtcagaatgccatttgcctg

SEQ ID NO: 10 - DNA, 1188 bp, synthetic construct
  misc_feature 1..1188: note = Description of sequence: alpha 1 antitrypsin ORF sequence
  source 1..1188: mol_type = other DNA
atggaagatccacaaggagatgctgcacaaaagactgacacctcccatcatgaccaggatcatccaacatttaacaaaatcacaccaaatcttgccgaattcgcattttcattgtatagacaattagctcatcaaagcaactccacaaacattttcttctcccctgtttccattgcaactgcttttgctatgctttcgttaggtacgaaagctgacacccatgatgaaatcctcgaagggttgaatttcaatttgaccgaaatccccgaggctcagattcacgaaggtttccaagagttgttgagaactttgaatcaaccggattctcagttacaactgactactggtaacggtttgttcttgagcgaaggattgaaactggttgacaaattccttgaggatgtcaaaaagttgtatcactcggaagcatttactgtgaatttcggagatactgaggaggccaaaaagcaaatcaatgactatgtcgaaaaaggtacacagggtaagattgttgatttggtgaaggaattggaccgtgatacggttttcgctctggttaactacatctttttcaagggtaaatgggagcgtccatttgaagtcaaagacactgaggaggaagatttccatgttgaccaggtaactacagtgaaagtaccaatgatgaaaagacttggaatgttcaacatccaacactgtaaaaagttgagttcatgggtattacttatgaaatacctgggaaatgctactgctatattctttctcccagacgagggcaaattacaacacctagagaatgaactgactcatgacattatcacaaagttcttggagaatgaggatagaaggagtgccagcttgcatttgcctaagctgtcaattacaggcacatacgacttgaaatccgtgctgggtcagctaggaattacaaaggtgttttctaatggtgcagacttatccggtgtcacggaggaggcacccctgaaactatctaaagcagttcacaaggctgtcctgaccattgatgaaaaaggaactgaggctgccggtgcaatgtttcttgaagctatcccaatgtcaataccacctgaagtgaagtttaacaagccttttgtttttctaatgatcgaacaaaacaccaagtctcctttgtttatgggtaaggttgttaatcctacacaaaagtaa

SEQ ID NO: 11 - DNA, 1212 bp, synthetic construct
  misc_feature 1..1212: note = Description of sequence: alpha 1 antitrypsin ORF sequence
  source 1..1212: mol_type = other DNA
atgcatcaccatcatcatcaccaccacgaagatccacaaggagatgctgcacaaaagactgacacctcccatcatgaccaggatcatccaacatttaacaaaatcacaccaaatcttgccgaattcgcattttcattgtatagacaattagctcatcaaagcaactccacaaacattttcttctcccctgtttccattgcaactgcttttgctatgctttcgttaggtacgaaagctgacacccatgatgaaatcctcgaagggttgaatttcaatttgaccgaaatccccgaggctcagattcacgaaggtttccaagagttgttgagaactttgaatcaaccggattctcagttacaactgactactggtaacggtttgttcttgagcgaaggattgaaactggttgacaaattccttgaggatgtcaaaaagttgtatcactcggaagcatttactgtgaatttcggagatactgaggaggccaaaaagcaaatcaatgactatgtcgaaaaaggtacacagggtaagattgttgatttggtgaaggaattggaccgtgatacggttttcgctctggttaactacatctttttcaagggtaaatgggagcgtccatttgaagtcaaagacactgaggaggaagatttccatgttgaccaggtaactacagtgaaagtaccaatgatgaaaagacttggaatgttcaacatccaacactgtaaaaagttgagttcatgggtattacttatgaaatacctgggaaatgctactgctatattctttctcccagacgagggcaaattacaacacctagagaatgaactgactcatgacattatcacaaagttcttggagaatgaggatagaaggagtgccagcttgcatttgcctaagctgtcaattacaggcacatacgacttgaaatccgtgctgggtcagctaggaattacaaaggtgttttctaatggtgcagacttatccggtgtcacggaggaggcacccctgaaactatctaaagcagttcacaaggctgtcctgaccattgatgaaaaaggaactgaggctgccggtgcaatgtttcttgaagctatcccaatgtcaataccacctgaagtgaagtttaacaagccttttgtttttctaatgatcgaacaaaacaccaagtctcctttgtttatgggtaaggttgttaatcctacacaaaagtaa

SEQ ID NO: 12 - DNA, 1212 bp, synthetic construct
  misc_feature 1..1212: note = Description of sequence: alpha 1 antitrypsin with C-term His Tag
  source 1..1212: mol_type = other DNA
atggaagatccacaaggagatgctgcacaaaagactgacacctcccatcatgaccaggatcatccaacatttaacaaaatcacaccaaatcttgccgaattcgcattttcattgtatagacaattagctcatcaaagcaactccacaaacattttcttctcccctgtttccattgcaactgcttttgctatgctttcgttaggtacgaaagctgacacccatgatgaaatcctcgaagggttgaatttcaatttgaccgaaatccccgaggctcagattcacgaaggtttccaagagttgttgagaactttgaatcaaccggattctcagttacaactgactactggtaacggtttgttcttgagcgaaggattgaaactggttgacaaattccttgaggatgtcaaaaagttgtatcactcggaagcatttactgtgaatttcggagatactgaggaggccaaaaagcaaatcaatgactatgtcgaaaaaggtacacagggtaagattgttgatttggtgaaggaattggaccgtgatacggttttcgctctggttaactacatctttttcaagggtaaatgggagcgtccatttgaagtcaaagacactgaggaggaagatttccatgttgaccaggtaactacagtgaaagtaccaatgatgaaaagacttggaatgttcaacatccaacactgtaaaaagttgagttcatgggtattacttatgaaatacctgggaaatgctactgctatattctttctcccagacgagggcaaattacaacacctagagaatgaactgactcatgacattatcacaaagttcttggagaatgaggatagaaggagtgccagcttgcatttgcctaagctgtcaattacaggcacatacgacttgaaatccgtgctgggtcagctaggaattacaaaggtgttttctaatggtgcagacttatccggtgtcacggaggaggcacccctgaaactatctaaagcagttcacaaggctgtcctgaccattgatgaaaaaggaactgaggctgccggtgcaatgtttcttgaagctatcccaatgtcaataccacctgaagtgaagtttaacaagccttttgtttttctaatgatcgaacaaaacaccaagtctcctttgtttatgggtaaggttgttaatcctacacaaaagcatcaccatcatcatcaccaccactaa

SEQ ID NO: 13 - DNA, 1600 bp, Homo sapiens; identical to SEQ ID NO: 6
  misc_feature 1..1600: note = Description of sequence: serpin family A member 1
  source 1..1600: mol_type = genomic DNA

SEQ ID NO: 14 - protein, 418 aa, Homo sapiens; identical to SEQ ID NO: 1
  REGION 1..418: note = Description of sequence: Alpha-1 antitrypsin precursor

SEQ ID NO: 15 - protein, 404 aa, Komagataella phaffii
  REGION 1..404: note = Description of sequence: WT OCH1 protein
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 16 - protein, 5 aa, synthetic construct
  REGION 1..5: note = Description of sequence: First five amino acids of OCH1 protein
FYMAI

SEQ ID NO: 17 - protein, 4 aa, synthetic construct
  REGION 1..4: note = Description of sequence: ER-retention signal
HDEL

SEQ ID NO: 18 - protein, 395 aa, synthetic construct
  REGION 1..395: note = Description of sequence: A1AT
MEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 19 - protein, 403 aa, synthetic construct
  REGION 1..403: note = Description of sequence: A1AT N-Terminal tag
MHHHHHHHHEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 20 - protein, 403 aa, synthetic construct
  REGION 1..403: note = Description of sequence: A1AT C-terminal tag
MEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKHHHHHHHH

SEQ ID NO: 21 - protein, 523 aa, synthetic construct
  REGION 1..523: note = Description of sequence: alpha-1,2-Mannosidase
MRFPSSSVLALGLIGPALAYPKPGATKRGSPNPTRAAAVKAAFQTSWNAYHHFAFPHDDLHPVSNSFDDERNGWGSSAIDGLDTAILMGDADIVNTILQYVPQINFTTTAVANQGSSVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLGADSTIGHLGSHPSTRKDLTFLSSYNGQSTSPNSGHLASFGGGNFILGGILLNEQKYIDFGIKLASSYFGTYTQTASGIGPEGFAWVDSVTGAGGSPPSSQSGFYSSAGFWVTAPYYILRPETLESLYYAYRVTGDSKWQDLAWEALSAIEDACRAGSAYSSINDVTQANGGGASDDMESFWFAEALKYAYLIFAEESDVQVQATGGNKFVFNTEAHPFSIRSSSRRGGHLA

SEQ ID NO: 22 - DNA, 1185 bp, synthetic construct
  misc_feature 1..1185: note = Description of sequence: alpha-1-antitrypsin precursor nucleotide sequence
  source 1..1185: mol_type = other DNA
gaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa